(19) United States
(12) Patent Application Publication
(10) Pub. No.: US 2006/0293268 A1
(43) Pub. Date: Dec. 28, 2006

(54) Antisense antiviral compounds and methods for treating foot and mouth disease

(76) Inventors: Aida E. Rieder, Westbrook, CT (US); David A. Stein, Corvallis, OR (US); Ariel E. Vagnozzi, Ituzaingo (AR); Dwight D. Weller, Corvallis, OR (US); Patrick L. Iversen, Corvallis, OR (US)

Correspondence Address: PERKINS COIE LLP, P.O. BOX 2168, MENLO PARK, CA, 94026, US

(21) Appl. No.: 11/418,904

(22) Filed: May 4, 2006

Related U.S. Application Data
(60) Provisional application No. 60/678,439, filed on May 5, 2005.

(51) Int. Cl. A61K 48/00; C07F 9/6533

(52) U.S. Cl. 514/44; 514/81; 544/81

(57) ABSTRACT

An antiviral antisense composition and method for treating foot-and-mouth disease virus (FMDV) in veterinary animals is disclosed. The composition contains an antisense compound that has a sequence effective to target at least 12 contiguous bases of an FMDV RNA sequence within a region of the positive-strand genomic RNA defined by SEQ ID NO: 25, and preferably, one of the viral sequences within SEQ ID NO:25 identified by SEQ ID NOS: 26-28. The composition is administered in a therapeutically effective amount in treating FMDV.

This application claims priority to U.S. provisional patent application No. 60/678,439 filed May 5, 2005, which is incorporated herein in its entirety by reference.

### FIELD OF THE INVENTION

This invention relates to antisense oligonucleotide compounds and methods for treating viral infections by foot and mouth disease virus.

### REFERENCES

Agrawal, S., S. H. Mayrand, et al. (1990). “Site-specific excision from RNA by RNase H and mixed-phosphate-backbone oligodeoxynucleotides.” Proc Natl Acad Sci USA 87(4): 1401-5.
Belsham, G. J. (2005). “Translation and replication of FMDV RNA.” Curr Top Microbiol Immunol 288: 43-70.
Blommers, M. J., U. Pieles, et al. (1994). “An approach to the structure determination of nucleic acid analogues hybridized to RNA. NMR studies of a duplex between 2′-OMe RNA and an oligonucleotide containing a single amide backbone modification.” Nucleic Acids Res 22(20): 4187-94.
Bonham, M. A., S. Brown, et al. (1995). “An assessment of the antisense properties of RNase H-competent and steric-blocking oligomers.” Nucleic Acids Res 23(7): 1197-203.
Boudvillain, M., M. Guerin, et al. (1997). “Transplatin-modified oligo(2′-O-methyl ribonucleotide)s: a new tool for selective modulation of gene expression.” Biochemistry 36(10): 2925-31.
Cao, X., I. E. Bergmann, et al. (1995). “Functional analysis of the two alternative translation initiation sites of foot-and-mouth disease virus.” J Virol 69(1): 560-3.
Cross, C. W., J. S. Rice, et al. (1997). “Solution structure of an RNA×DNA hybrid duplex containing a 3′-thioformacetal linker and an RNA A-tract.” Biochemistry 36(14): 4096-107.
Dagle, J. M., J. L. Littig, et al. (2000). “Targeted elimination of zygotic messages in Xenopus laevis embryos by modified oligonucleotides possessing terminal cationic linkages.” Nucleic Acids Res 28(10): 2153-7.
Egholm, M., O. Buchardt, et al. (1993). “PNA hybridizes to complementary oligonucleotides obeying the Watson-Crick hydrogen-bonding rules.” Nature 365(6446): 566-8.
Felgner, P. L., T. R. Gadek, et al. (1987). “Lipofection: a highly efficient, lipid-mediated DNA-transfection procedure.” Proc Natl Acad Sci USA 84(21): 7413-7.
Gait, M. J., A. S. Jones, et al. (1974). “Synthetic-analogues of polynucleotides XII. Synthesis of thymidine derivatives containing an oxyacetamido- or an oxyformamido-linkage instead of a phosphodiester group.” J Chem Soc [Perkin 1] 0(14): 1684-6.
Grubman, M. J. and B. Baxt (2004). “Foot-and-mouth disease.” Clin Microbiol Rev 17(2): 465-93.
Lesnikowski, Z. J., M. Jaworska, et al. (1990). “Octa(thymidine methanephosphonates) of partially defined stereochemistry: synthesis and effect of chirality at phosphorus on binding to pentadecadeoxyriboadenylic acid.” Nucleic Acids Res 18(8): 2109-15.
Mahy, B. W. (2005). “Introduction and history of foot-and-mouth disease virus.” Curr Top Microbiol Immunol 288: 1-8.
Mertes, M. P. and E. A. Coats (1969). “Synthesis of carbonate analogs of dinucleosides. 3′-Thymidinyl 5′-thymidinyl carbonate, 3′-thymidinyl 5′-(5-fluoro-2′-deoxyuridinyl)carbonate, and 3′-(5-fluoro-2′-deoxyuridinyl) 5′-thymidinyl carbonate.” J Med Chem 12(1): 154-7.
Moulton, H. M., M. H. Nelson, et al. (2004). “Cellular uptake of antisense morpholino oligomers conjugated to arginine-rich peptides.” Bioconjug Chem 15(2): 290-9.
Strauss, J. H. and E. G. Strauss (2002). Viruses and Human Disease. San Diego, Academic Press.
Summerton, J. and D. Weller (1997). “Morpholino antisense oligomers: design, preparation, and properties.” Antisense Nucleic Acid Drug Dev 7(3): 187-95.

### BACKGROUND OF THE INVENTION

Foot-and-Mouth Disease (FMD) is a highly contagious, severely debilitating disease that infects all cloven-hoofed animals. It is endemic in many developing countries worldwide. In particular, swine in Asia are frequently affected by FMD. An epidemic of FMD reduces livestock productivity, leads to high vaccination costs, and restricts the international trade of livestock and livestock products. Economically, FMD is the most important animal disease of livestock worldwide.

FMD disease is caused by a member of the family Picornaviridae, genus Aphtovirus, foot-and-mouth disease virus (FMDV), a small virus having a single stranded positive sense RNA genome of about 8,000 nucleotides. As is the case with other small RNA viruses, FMDV is genetically and antigenically variable, with seven different serotypes and tens of subtypes causing outbreaks in endemic areas around the world. FMD is characterized by debilitating oral and pedal vesicles, which can result in a significant decline in production of meat or dairy products, but generally low mortality. However, in young animals, infection of the heart muscle may result in severe myocardial necrosis and death. FMD is listed in the World Organization for Animal Health (OIE) List A of reportable diseases and its occurrence in a country results in immediate restrictions for trade of animal and animal products to other FMD-free countries. The disease does not occur in the US, Canada, or Mexico, and its continued absence from North America is a priority for the US livestock industry as it allows trading of animals and animal products with other FMD-free countries.

FMDV is perhaps most contagious pathogen known and spread of the virus is rapid and requires rapid interventions (such as quarantines and destruction of infected animals) in order to limit and control outbreaks. FMD can be spread by contact, aerosol or through movement of animals or animal products, and personnel. The alarming rate of spread, as recently demonstrated during an outbreak in Taiwan in the spring of 1997 and in the devastating outbreak in the UK in 2001, makes it very difficult and costly to control FMD outbreaks. These outbreaks cost the economies of these countries billions of dollars, not only in direct costs to the animal industry, but also in tourism (due to quarantines), animal feed and pharmaceutical industries among others. Because of the highly infectious nature of FMD, countries that do not have the disease maintain rigid quarantine and import restrictions on animals and animal products from infected countries to prevent its introduction and allow their active participation in international trade. Currently, when outbreaks occur in FMD-free countries, control is attempted by stopping animal movement, destruction of animals in affected and neighboring premises, disinfection, and ring vaccination using a serotype-specific killed vaccine. Over four million animals, most of which not infected by FMDV, were destroyed before the 2001 outbreak in the UK was controlled. Current inactivated whole virus vaccines used in FMD control have several shortcomings; production requires growing large quantities of virulent FMDV in BL-3 containment facilities, vaccines are serotype specific and in some cases, cross protection is not achieved even within the same serotype. In some cases, protection is not achieved until at least 7-14 days post vaccination. In addition, vaccination does not prevent infection in all cases resulting in healthy carrier animals and it is difficult to distinguish vaccinated from infected animals. Because of these shortcomings FMD-free countries hesitate to use vaccination during outbreaks. On the other hand, the mass destruction of animals with pyres of burning livestock in the UK countryside dominating the news has resulted in strong public outcry and opposition to such measures to control FMD outbreaks in the future.

Currently, the US maintains the North American FMD Vaccine Bank at the Plum Island Animal Disease Center (PIADC). This vaccine antigen is purchased from foreign countries, since Federal law only allows FMDV at PIADC, and consists of reserves of antigen for the seven serotypes of FMDV. Vaccine is made available for an outbreak in the US, Canada, or Mexico, but must be formulated by the manufacturer (currently in the UK) in the event of an emergency. Scientists at ARS-PIADC Foreign Animal Disease Research program have recently demonstrated that current inactivated vaccines can induce protection as early as seven days post vaccination but this might not be fast enough to contain the spread of FMDV.

Despite the considerable socio-economic impact of the pathogenic Aphthoviruses there is no effective antiviral drug therapy currently available and so far only vaccine-based strategies have been effectively applied to control FMD in endemic and non-endemic areas. New antiviral drugs are needed for the early treatment of FMDV infections in the face of an outbreak because, unlike vaccines, antiviral drugs can block infection after it has started, something a vaccine cannot do immediately.

Based on the above, there is an unmet need for the development of rapid-acting antiviral compounds capable of providing immediate protection against Aphthoviruses and, in particular, various serotypes of FMDV, to prevent infection, carrier state and viral shedding, that can be easily delivered and provide protection while vaccine-induced innate responses occur.

### SUMMARY OF THE INVENTION

The invention includes, in one aspect, an antiviral antisense composition for inhibiting replication within a host cell of foot-and-mouth disease virus (FMDV). The composition includes an oligonucleotide analog compound characterized by:

(i) a nuclease-resistant backbone,

(ii) capable of uptake by mammalian host cells,

(iii) containing between 15-40 nucleotide bases,

(iv) having a targeting sequence that is complementary to a target sequence composed of at least 12 contiguous bases within the positive-strand FMDV RNA sequence defined by SEQ ID NO:25,

(v) an ability to form with the RNA target sequence, a heteroduplex structure (a) composed of the target region of the positive sense strand of the virus and the oligonucleotide compound, and (b) characterized by a Tm of dissociation of at least 45° C.; and

(vi) an ability, at a concentration of 2.5 μM, to reduce the viral titre in cultured BHK-21 cells infected with 0.5 PFU/cell of A24 Cruzeiro strain of FMDV, at least 4 orders of magnitude, and up to 6 orders of magnitude or more.

The compound may be composed of morpholino subunits linked by uncharged, phosphorus-containing intersubunit linkages, joining a morpholino nitrogen of one subunit to a 5′ exocyclic carbon of an adjacent subunit. In one embodiment, the intersubunit linkages are phosphorodiamidate linkages, such as those having the structure: 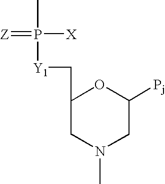
where Y₁=O, Z=O, Pj is a purine or pyrimidine base-pairing moiety effective to bind, by base-specific hydrogen bonding, to a base in a polynucleotide, and X is alkyl, alkoxy, thioalkoxy, or an alkyl amino or an alkyl amino of the form wherein NR₂, where each R is independently hydrogen or methyl.

The compound may be composed of morpholino subunits linked with the uncharged linkages described above interspersed with linkages that are positively charged at physiological pH. The total number of positively charged linkages is between 2 and no more than half of the total number of linkages. The positively charged linkages have the structure above, where X is 1-piperazine.

The compound may be a covalent conjugate of an oligonucleotide analog moiety capable of forming such a heteroduplex structure with the positive or negative sense strand of the virus, and an arginine-rich polypeptide effective to enhance the uptake of the compound into host cells. 7. Exemplary arginine-rich polypeptides have one of the sequences identified by SEQ ID NOS: 33-35.

The compounds may have a sequence effective to target at least 12 contiguous bases of one of the sequences identified by SEQ ID NOS: 26-28. Exemplary compound sequences at least 15 contiguous bases of a sequence selected from the group consisting of SEQ ID NOS; 29-32, such as the compound sequences identified by SEQ ID NOS; 29-32, or SEQ ID NOS: 11-13.

In another aspect, the invention includes a method of treating a FMDV infection in a mammalian host, by administering to the host, a therapeutically effective amount of a composition of the type described above, including the exemplary compositions.

These and other objects and features of the invention will become more fully apparent when the following detailed description of the invention is read in conjunction with the accompanying drawings.

### BRIEF DESCRIPTION OF THE FIGURES

FIGS. 1A-1D show the repeating subunit segment of exemplary morpholino oligomers;

FIGS. 2A-2G show the backbone structures of various oligonucleotide analogs with uncharged backbones. FIG. 2H shows the structure of a preferred cationic linkage;

FIG. 3 illustrate the arrangement of FMDV genes in the viral genome and RNA structural elements in the 5′ and 3′-UTRs (from Grubman and Baxt 2004);

FIG. 4 shows the target regions of six exemplary antisense compounds targeted against FMDV in the context of the predicted secondary structure of the 5′ and 3′ UTRs;

FIG. 5 shows FMDV A24 Cruzeiro replication under different antiviral PMO treatments compared to controls;

FIG. 6 shows the specificity of PMOs to inhibit FMDV replication and not to bovine enterovirus (BEV);

FIG. 7 shows that the IRES 5D PMO (SEQ ID NO:11) inhibits replication of multiple FMDV serotypes.

FIG. 8 shows a generalized PMO structure with an arginine-rich peptide conjugated to the 5′ terminus. Single letter codes for amino acids are used expect for the non-natural amino acids beta-alanine (β-Ala) and 6-aminohexanoic acid (Ahx).

FIG. 9 shows the sequence alignment of target regions of eight different FMDV serotypes.

FIG. 10 shows the synthetic steps to produce subunits used to produce +PMO containing the (1-piperazino)phosphinylideneoxy cationic linkage as shown in FIG. 2H.

### DETAILED DESCRIPTION OF THE INVENTION

I. Definitions

The terms below, as used herein, have the following meanings, unless indicated otherwise:

The terms “oligonucleotide analog” refers to an oligonucleotide having (i) a modified backbone structure, e.g., a backbone other than the standard phosphodiester linkage found in natural oligo- and polynucleotides, and (ii) optionally, modified sugar moieties, e.g., morpholino moieties rather than ribose or deoxyribose moieties. The analog supports bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Preferred analogs are those having a substantially uncharged, phosphorus containing backbone.

A substantially uncharged, phosphorus containing backbone in an oligonucleotide analog is one in which a majority of the subunit linkages, e.g., between 50-100%, are uncharged at physiological pH, and contain a single phosphorous atom. The analog contains between 12 and 40 subunits, typically about 15-25 subunits, and preferably about 18 to 25 subunits. The analog may have exact sequence complementarity to the target sequence or near complementarity, as defined below.

A “subunit” of an oligonucleotide analog refers to one nucleotide (or nucleotide analog) unit of the analog. The term may refer to the nucleotide unit with or without the attached intersubunit linkage, although, when referring to a “charged subunit”, the charge typically resides within the intersubunit linkage (e.g. a phosphate or phosphorothioate linkage).

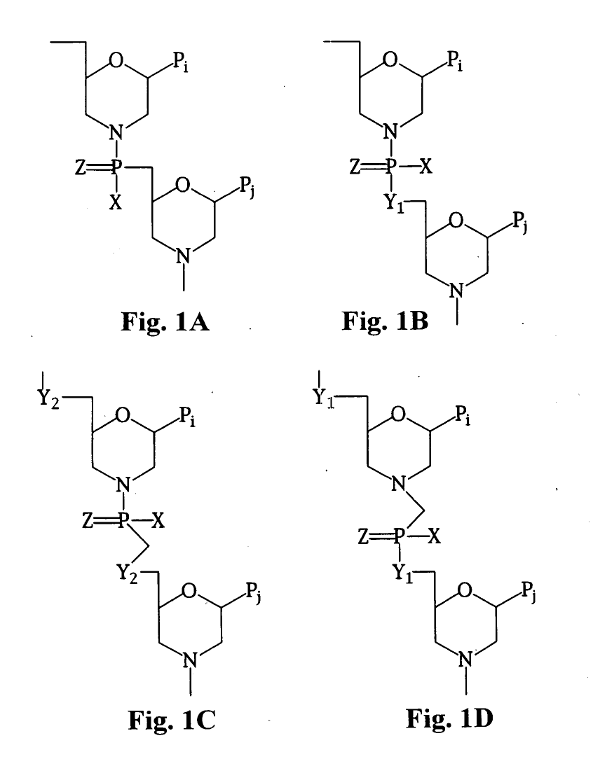

A “morpholino oligonucleotide analog” is an oligonucleotide analog composed of morpholino subunit structures of the form shown in FIGS. 1A-1D, where (i) the structures are linked together by phosphorus-containing linkages, one to three atoms long, joining the morpholino nitrogen of one subunit to the 5′ exocyclic carbon of an adjacent subunit, and (ii) Pᵢ and Pⱼ are purine or pyrimidine base-pairing moieties effective to bind, by base-specific hydrogen bonding, to a base in a polynucleotide. The purine or pyrimidine base-pairing moiety is typically adenine, cytosine, guanine, uracil or thymine. The synthesis, structures, and binding characteristics of morpholino oligomers are detailed in U.S. Pat. Nos. 5,698,685, 5,217,866, 5,142,047, 5,034,506, 5,166,315, 5,521,063, and 5,506,337, all of which are incorporated herein by reference.

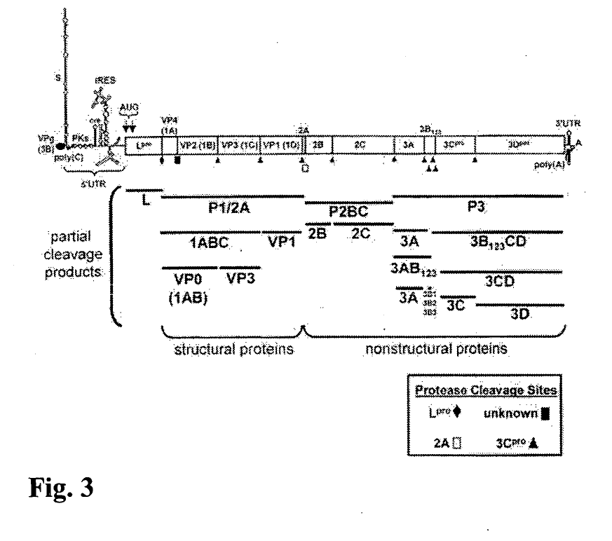

The subunit and linkage shown in FIG. 1B are used for six-atom repeating-unit backbones, as shown in FIG. 3B (where the six atoms include: a morpholino nitrogen, the connected phosphorus atom, the atom (usually oxygen) linking the phosphorus atom to the 5′ exocyclic carbon, the 5′ exocyclic carbon, and two carbon atoms of the next morpholino ring). In these structures, the atom Y₁ linking the 5′ exocyclic morpholino carbon to the phosphorus group may be sulfur, nitrogen, carbon or, preferably, oxygen. The X moiety pendant from the phosphorus is any stable group which does not interfere with base-specific hydrogen bonding. Preferred X groups include fluoro, alkyl, alkoxy, thioalkoxy, and alkyl amino, including cyclic amines, all of which can be variously substituted, as long as base-specific bonding is not disrupted. Alkyl, alkoxy and thioalkoxy preferably include 1-6 carbon atoms. Alkyl amino preferably refers to lower alkyl (C₁ to C₆) substitution, and cyclic amines are preferably 5- to 7-membered nitrogen heterocycles optionally containing 1-2 additional heteroatoms selected from oxygen, nitrogen, and sulfur. Z is sulfur or oxygen, and is preferably oxygen.

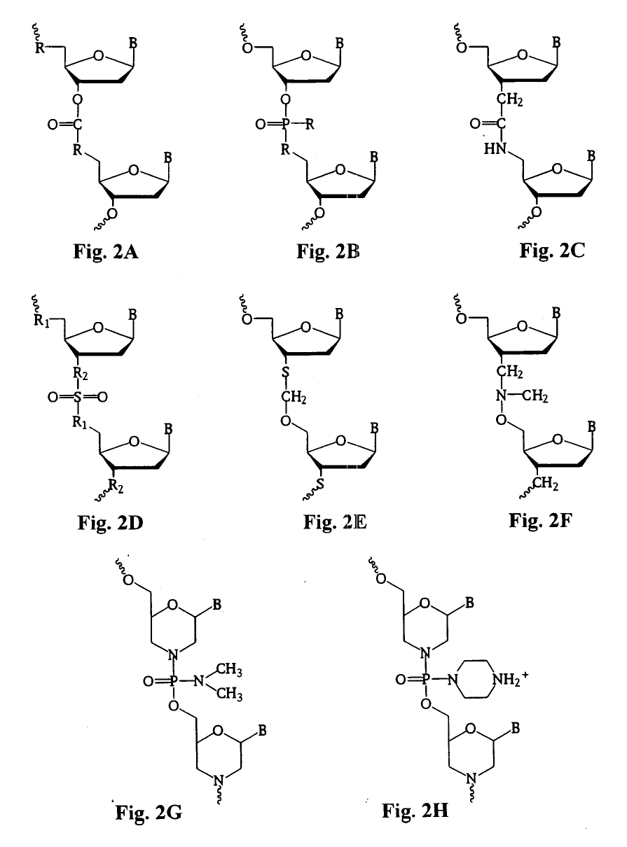

A preferred morpholino oligomer is a phosphorodiamidate-linked morpholino oligomer, referred to herein as a PMO. Such oligomers are composed of morpholino subunit structures such as shown in FIG. 1B, where X=NH₂, NHR, or NR₂ (where R is lower alkyl, preferably methyl), Y=O, and Z=O, and Pᵢ and Pⱼ are purine or pyrimidine base-pairing moieties effective to bind, by base-specific hydrogen bonding, to a base in a polynucleotide. Also preferred are structures having an alternate phosphorodiamidate linkage, where, in FIG. 1B, X=lower alkoxy, such as methoxy or ethoxy, Y=NH or NR, where R is lower alkyl, and Z=O. Also preferred are morpholino oligomers where the phosphordiamidate linkages are a mixture of uncharged linkages as shown in FIG. 2G and cationic linkages as shown in FIG. 2H where, in FIG. 1B, X=1-piperazino.

The term “substituted”, particularly with respect to an alkyl, alkoxy, thioalkoxy, or alkylamino group, refers to replacement of a hydrogen atom on carbon with a heteroatom-containing substituent, such as, for example, halogen, hydroxy, alkoxy, thiol, alkylthio, amino, alkylamino, imino, oxo (keto), nitro, cyano, or various acids or esters such as carboxylic, sulfonic, or phosphonic. It may also refer to replacement of a hydrogen atom on a heteroatom (such as an amine hydrogen) with an alkyl, carbonyl or other carbon containing group.

As used herein, the term “target sequence” refers to a target sequence composed of at least 12 contiguous bases within the positive-strand FMDV RNA sequence defined by SEQ ID NO:25, or any serotype-specific homologous sequence, such as those identified as SEQ ID NO: 7, and SEQ ID NOS: 18-25, and may include, without limitation, (i) the AUG1 start site region of the FMDV polyprotein identified by SEQ ID NOS:27 or a homologous serotype-specific sequences such as SEQ ID NO: 4; (ii) the AUG2 start site region of the FMDV polyprotein identified by SEQ ID NOS:28 or a homologous serotype-specific sequences such as SEQ ID NO: 5 or (iii) the IRES region of the FMDV viral RNA identified by SEQ ID NOS: 26 or a homologous serotype-specific sequences such as SEQ ID NO: 3. The “target sequence” refers to a portion of the target RNA against which the oligonucleotide analog is directed, that is, the sequence to which the oligonucleotide analog will hybridize by Watson-Crick base pairing of a complementary sequence.

The term “targeting sequence” is the sequence in the oligonucleotide compound that is complementary (meaning, in addition, substantially complementary) to the target sequence in the RNA genome. The entire sequence, or only a portion, of the analog compound may be complementary to the target sequence. For example, in a compound having 20 bases, only 12-14 bases may be targeting sequences.

Target and targeting sequences are described as “complementary” to one another when hybridization occurs in an antiparallel configuration. A targeting sequence may have “near” or “substantial” complementarity to the target sequence and still function for the purpose of the present invention, that is, still be “complementary.” Preferably, the oligonucleotide analog compounds employed in the present invention have at most one mismatch with the target sequence out of 10 nucleotides, and preferably at most one mismatch out of 20. Alternatively, the antisense oligomers employed have at least 90% sequence homology, and preferably at least 95% sequence homology, with the exemplary targeting sequences as designated herein.

An oligonucleotide analog “specifically hybridizes” to a target polynucleotide if the oligomer hybridizes to the target under physiological conditions, with a Tₘ substantially greater than 45° C., preferably at least 50° C., and typically 60° C.-80° C. or higher. Such hybridization preferably corresponds to stringent hybridization conditions. At a given ionic strength and pH, the Tₘ is the temperature at which 50% of a target sequence hybridizes to a complementary polynucleotide. Again, such hybridization may occur with “near” or “substantial” complementary of the antisense oligomer to the target sequence, as well as with exact complementarity.

A “nuclease-resistant” oligomeric molecule (oligomer) refers to one whose backbone is substantially resistant to nuclease cleavage, in non-hybridized or hybridized form; by common extracellular and intracellular nucleases in the body, that is, the oligomer shows little or no nuclease cleavage under normal nuclease conditions in the body to which the oligomer is exposed

A “heteroduplex” refers to a duplex between an oligonucleotide compound and the complementary portion of a target RNA. A “nuclease-resistant heteroduplex” refers to a heteroduplex formed by the binding of an antisense oligomer to its complementary target, such that the heteroduplex is substantially resistant to in vivo degradation by intracellular and extracellular nucleases, such as RNAseH, which are capable of cutting double-stranded RNA/RNA or RNA/DNA complexes.

A “base-specific intracellular binding event involving a target RNA” refers to the specific binding of an oligonucleotide analog to a target RNA sequence inside a cell. The base specificity of such binding is sequence specific. For example, a single-stranded polynucleotide can specifically bind to a single-stranded polynucleotide that is complementary in sequence.

An “effective amount” of an antisense oligomer, targeted against an infecting RNA virus, is an amount effective to reduce the rate of replication of the infecting virus, and/or viral load, and/or symptoms associated with the viral infection.

As used herein, the term “body fluid” encompasses a variety of sample types obtained from a subject including, urine, saliva, plasma, blood, spinal fluid, or other sample of biological origin, such as skin cells or dermal debris, and may refer to cells or cell fragments suspended therein, or the liquid medium and its solutes.

The term “relative amount” is used where a comparison is made between a test measurement and a control measurement. The relative amount of a reagent forming a complex in a reaction is the amount reacting with a test specimen, compared with the amount reacting with a control specimen. The control specimen may be run separately in the same assay, or it may be part of the same sample (for example, normal tissue surrounding a malignant area in a tissue section).

“Treatment” of an individual or a cell is any type of intervention provided as a means to alter the natural course of the individual or cell. Treatment includes, but is not limited to, administration of e.g., a pharmaceutical composition, and may be performed either prophylactically, or subsequent to the initiation of a pathologic event or contact with an etiologic agent. The related term “improved therapeutic outcome” relative to a patient diagnosed as infected with a particular virus, refers to a slowing or diminution in the growth of virus, or viral load, or detectable symptoms associated with infection by that particular virus.

An agent is “actively taken up by mammalian cells” when the agent can enter the cell by a mechanism other than passive diffusion across the cell membrane. The agent may be transported, for example, by “active transport”, referring to transport of agents across a mammalian cell membrane by e.g. an ATP-dependent transport mechanism, or by “facilitated transport”, referring to transport of antisense agents across the cell membrane by a transport mechanism that requires binding of the agent to a transport protein, which then facilitates passage of the bound agent across the membrane. For both active and facilitated transport, the oligonucleotide analog preferably has a substantially uncharged backbone, as defined below. Alternatively, the antisense compound may be formulated in a complexed form, such as an agent having an anionic backbone complexed with cationic lipids or liposomes, which can be taken into cells by an endocytotic mechanism. The analog also may be conjugated, e.g., at its 5′ or 3′ end, to an arginine-rich peptide, e.g., a portion of the HIV TAT protein, or polyarginine, to facilitate transport into the target host cell as described (Moulton, Nelson et al. 2004). Exemplary arginine-rich delivery peptides are shown in the sequence listing as SEQ ID NOS: 18-20.

II. Targeted Viruses

The present invention is based on the discovery that effective inhibition of FMDV can be achieved by exposing cells infected with the virus to an antisense oligonucleotide compound (i) targeted against a target sequence composed of at least 12 contiguous bases within the positive-strand FMDV RNA sequence defined by SEQ ID NO:25, or, where the particular infecting serotype has been identified, targeted against the homologous sequence for that serotype, such as the serotype-specific sequences identified as SEQ ID NO: 7, and SEQ ID NOS: 18-25. The target sequence may include, without limitation, (i) the AUG1 start site region of the FMDV polyprotein identified by SEQ ID NOS:27 or a homologous serotype-specific sequence such as SEQ ID NO: 4; (ii) the AUG2 start site region of the FMDV polyprotein identified by SEQ ID NOS:28 or a homologous serotype-specific sequence such as SEQ ID NO: 5 or (iii) the IRES region of the FMDV viral RNA identified by SEQ ID NOS: 26 or a homologous serotype-specific sequences such as SEQ ID NO: 3. and (ii) having physical and pharmacokinetic features which allow effective interaction between the antisense compound and the virus within host cells. In one aspect, the oligomers can be used in treating a mammalian subject infected with the virus.

The invention targets FMDV viruses having RNA genomes that are: (i) single stranded, (ii) positive polarity, and (iii) less than 12 kb. The targeted viruses also synthesize an RNA species with negative polarity, the negative-strand or (−)RNA, as the requisite step in viral gene expression. Various physical, morphological, and biological characteristics of the FMDV can be found, for example, in Fields Virology and “Viruses and Human Disease” (Strauss and Strauss 2002) and at the Universal Virus Database of the International Committee on Taxonomy of Viruses (http://www.ncbi.nlm.nih.gov/ICTVdb/index.htm). Seven serotypes of FMDV have been identified to date. The virus structure is a roughly spherical particle with a sedimentation coefficient of 140S. The viral particle measure approximately 25 nm in diameter and contains one, single-stranded, positive polarity viral RNA genome. Recent reviews provide extensive background into the molecular biology, pathogenesis, disease control measures and recent outbreaks of FMD and FMDV and are incorporated herein in their entirety (Grubman and Baxt 2004; Belsham 2005; Mahy 2005).

B. Target Sequences

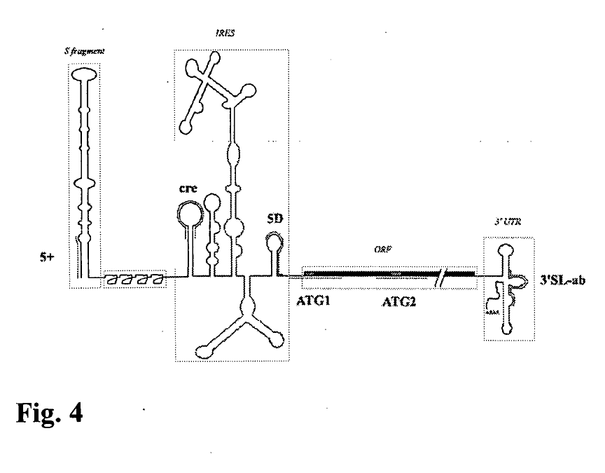

The FMDV virus genome is approximately 8,300 bases of single-stranded RNA that is unsegmented and in the positive-sense orientation. The FMDV genome consists of a single large open reading frame that encodes a single polyprotein which is immediately processed by virus encoded proteases during synthesis. The FMDV viral genome organization has been extensively reviewed (Grubman and Baxt 2004; Belsham 2005) and a diagram is provided in FIG. 3. The viral genome has some similarities to most cellular mRNAs in that it encodes a single long open reading frame of approximately 7000 nucleotides followed by a 3′ UTR of about 100 nucleotides and a poly(A) tail. However, FMDV is distinct from cellular mRNAs in that it has a very long, uncapped 5′ UTR of about 1200 nucleotides which is somewhat longer that the typical 5′ UTR of other members of the Picornaviridae family. As shown in FIG. 4, the 5′ UTR is predicted to fold into a series of stem loop structures which are thought to be involved in both replication and translation of the polyprotein. One region serves as an internal ribosome entry site (IRES) element that directs cap-independent translation of the FMDV polyprotein (Belsham 2005). Either of two AUG start codons may be used during translation of the viral RNA, although only the second or downstream AUG start codon (AUG2) at position 1133 has been shown through mutational analysis to be required for viral replication (Cao, Bergmann et al. 1995).

The targets selected were positive-strand (sense) RNA sequences that (i) span or are just downstream or upstream (within 25 bases) of either of the two AUG start codons of FMDV virus proteins, (ii) the 5′ terminal 30 bases of the positive-strand viral RNA, (iii) a stem loop structure at the 3′ terminus of the positive strand, (iv) the CRE region in the 5′ UTR and (v) the IRES 5D element in the 5′ UTR. Of these, targets (i) and (v), both of which are in the region defined by SEQ ID NO: 25, showed unexpectedly high activity in inhibiting FMDV replication in an animal host, and 5-6 orders of magnitude greater inhibition than any of the other targets.

FMDV genome sequences can be obtained from GenBank using techniques well known in the art. The particular targeting sequences shown below and identified as SEQ ID NOS: 1-7 were selected for specificity against the FMDV-A24 Cruzeiro serotype (GenBank Acc. No. AY593768) for experimental reasons. Corresponding sequences for FMDV GenBank Reference genotypes and other FMDV genotypes are readily determined from the known GenBank entries for these viruses Exemplary FMDV genotypes and their GenBank numbers are: A12 Valle, GenBank Acc. No. AY593752; C3 Resende, GenBank Acc. No. AY593768; O1 Campos, GenBank Acc. No. AY593818; O1 Taiwan 99, GenBank Acc. No. AJ539136; Asia 1 GenBank Acc. No. AY593795; SAT 1 GenBank Acc. No. AF056511; SAT 2, GenBank Acc. No. AF540910. Preferably, targeting sequences are selected that give a maximum consensus among the different genotypes or base mismatches that can be accommodated by ambiguous bases in the antisense sequence, according to well-known base pairing rules.

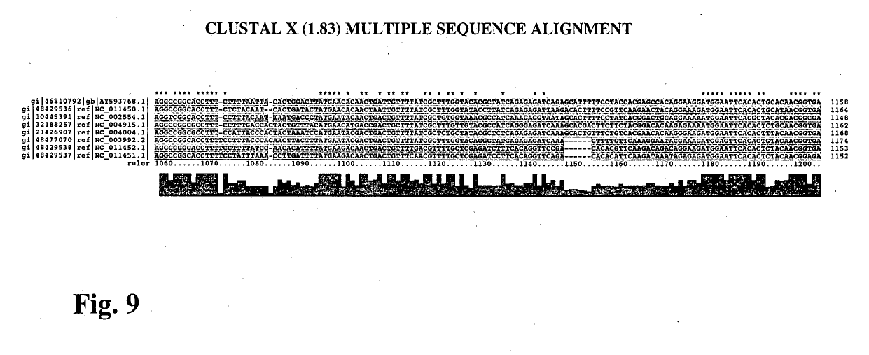

To illustrate the above, and in order to define a target sequence that applies to a number of different known serotypes of FMDV, seven other serotypes, in addition to the above A24 Cruziero serotype were analyzed. These serotypes are identified by GenBank accession numbers NC_{—}011450.1, NC_{—}002554.1, NC_{—}004915.1, NC_{—}004004.1, NC_{—}003992.2, NC_{—}011452.1, and NC_{—}011451.1. For each serotype, the sequence region corresponding to sequence region 1015-1158 (SEQ ID NO:7) of the A24 serotype, and containing the IRES D5 and AUG1 and AUF2 regions of the genome, was identified, and these additional sequences are identified by SEQ ID NOS: 18-24. The eight sequence regions (SEQ ID NOS: 7, and 18-24) were aligned using a standard alignment algorithm, with the results shown in FIG. 9. A consensus sequence for entire target region shown for each of the eight serotypes in FIG. 9 was generated (including SEQ ID NO: 7), and is represented by SEQ ID NO: 25, where G/A variations among the serotype sequences at any position are indicated by “R,” C/T variations by “C,” and variations involving both purine and pyrimidine bases, by “N.” The rationale of using the “C” instead of “Y” to designate a C/T variation is that in the targeting sequence, G will bind to either C or U in the RNA target. The same convention is used in SEQ ID NOS: 25-28. As seen, the aligned sequences have three regions of relatively greater consensus sequence, corresponding to target regions surrounding the IRES D5, AUG1 and AUG2 start sites of the genome, and these three regions are indicated by underlining in the figure. The consensus sequence for each of these regions (from SEQ ID NO: 25) is identified as SEQ ID NO: 26, 27, and 28, respectively.

GenBank references for exemplary viral nucleic acid target sequences representing FMDV genomic segments are listed in Table 1 below. It will be appreciated that these sequences are only illustrative of other serotypes of the FMDV as may be available from available gene-sequence databases of literature or patent resources (See e.g. http://www.ncbi.nlm.nih.gov/). The sequences in Table 1, identified as SEQ ID NOS: 1-7 and 25-28, are also listed in the Sequence Listing at the end of the specification. The target sequences in Table 1 identified as SEQ ID NOS: 1-7 represent selected regions of the 5′UTR of the positive strand RNA, the region from 25 bases upstream of AUG1 to 25 bases downstream of AUG2 codons of the polyprotein (AUG1-2; SEQ ID NO:7), and a region predicted to form a stem-loop at the 3′ terminus of the viral RNA (3′SLab; SEQ ID NO:6). The sequences shown are the positive-strand sequence in the 5′ to 3′ orientation.

TABLE 1

Exemplary FMCV Nucleic Acid Target
Sequences*

SEQ

| | | | |
|---|---|---|---|
| GenBank | Nucleotide | Sequence | ID |

| | | | | |
|---|---|---|---|---|
| Name | No. | Region | (5′ to 3′) | NO |

| | | | | |
|---|---|---|---|---|
| 233223777 | AY593768 | 1-21 | TTGAAAGGGGGCGCTAGG | 1 |
| | | | GTT | |
| CRE | AY593768 | 569-589 | CTTGTACAAACACGATCT | 2 |
| | | | AAG | |
| IRES D5 | AY593768 | 1015-1035 | AGGCCGGCACCTTTCTTT | 3 |
| | | | TAA | |
| AUG1 | AY593768 | 1036-1056 | TTACACTGGACTTATGAA | 4 |
| | | | CAC | |
| AUG1 | AY593768 | 1121-1141 | GCCACAGGAAGGATGGAA | 5 |
| | | | TTC | |
| 3′ SLab | AY593768 | 8085-8105 | GGCGCGCGACGCCGTAGG | 6 |
| | | | AGT | |
| AUG1-2 | AY593768 | 1015-1158 | AGGCCGGCACCTTTCTTT | 7 |
| | | | TAATTACACTGGACTTAT | |
| | | | GAACACAACTGATTGTTT | |
| | | | TATCGCTTTGGTACACGC | |
| | | | TATCAGAGAGATCAGAGC | |
| | | | ATTTTTCCTACCACGAGC | |
| | | | CACAGGAAGGATGGAATT | |
| | | | CACACTGCACAACGGTGA | |

| | | |
|---|---|---|
| Synthetic Consensus | GACCGGAGGCCGGCRCCT | 25 |
| Sequence | TTCCCTTAATTACACTGG | |
| | ACCCATGAANACRACTGA | |
| | CTGTTTTATCGCTNTGGT | |
| | ACACGCTATCAGAGAGAT | |
| | CAGAGCATTTTTCCTACC | |
| | ACGAGCCACARGRAARAT | |
| | GGAATTCACACTGCACAA | |
| | CGGTGA | |
| Synthetic Consensus Target | GACCGGAGGCCGGCRCCT | 26 |
| Sequence IRES | TTCCCTT | |
| Synthetic Consensus Target | CCCATGAANACRACTGAC | 27 |
| Sequence AUG1 | TGTTTTATCGCTNTG | |
| Synthetic Consensus Target | RGRAARATGGAATTCACA | 28 |
| Sequence AUG2 | CT | |

*R is Purine = A or G and N is G, A, C, or T

Targeting sequences are designed to hybridize to a region of the target sequence as listed in Table 1. Selected targeting sequences can be made shorter, e.g., 12 bases, or longer, e.g., 40 bases, and include a small number of mismatches, as long as the sequence is sufficiently complementary to disrupt the targeted stem structure(s) or translational initiation upon hybridization with the target, and forms with the virus positive-strand, a heteroduplex having a Tₘ of 45° C. or greater.

More generally, the degree of complementarity between the target and targeting sequence is sufficient to form a stable duplex. The region of complementarity of the antisense oligomers with the target RNA sequence may be as short as 8-11 bases, but is preferably 12-15 bases or more, e.g. 12-20 bases, or 12-25 bases. An antisense oligomer of about 14-15 bases is generally long enough to have a unique complementary sequence in the viral genome. In addition, a minimum length of complementary bases will be required to achieve the requisite binding Tₘ, as discussed below.

Oligomers as long as 40 bases may be suitable, where at least a minimum number of bases, e.g., 12 bases, are complementary to the target sequence. In general, however, facilitated or active uptake in cells is optimized at oligomer lengths less than about 30, preferably less than 25. For PMO oligomers, described further below, an optimum balance of binding stability and uptake generally occurs at lengths of 15-22 bases.

The oligomer may be 100% complementary to the viral nucleic acid target sequence, or it may include mismatches, e.g., to accommodate variants, as long as a heteroduplex formed between the oligomer and viral nucleic acid target sequence is sufficiently stable to withstand the action of cellular nucleases and other modes of degradation which may occur in vivo. Oligomer backbones which are less susceptible to cleavage by nucleases are discussed below. Mismatches, if present, are less destabilizing toward the end regions of the hybrid duplex than in the middle. The number of mismatches allowed will depend on the length of the oligomer, the percentage of G:C base pairs in the duplex, and the position of the mismatch(es) in the duplex, according to well understood principles of duplex stability. Although such an antisense oligomer is not necessarily 100% complementary to the viral nucleic acid target sequence, it is effective to stably and specifically bind to the target sequence, such that a biological activity of the nucleic acid target, e.g., expression of viral protein(s), is modulated.

The stability of the duplex formed between the oligomer and the target sequence is a function of the binding Tₘ and the susceptibility of the duplex to cellular enzymatic cleavage. The Tₘ of an antisense compound with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligonucleotide hybridization techniques. Methods Enzymol. Vol. 154 pp. 94-107. Each antisense oligomer should have a binding Tₘ, with respect to a complementary-sequence RNA, of greater than body temperature and preferably greater than 50° C. Tₘ's in the range 60-80° C. or greater are preferred. According to well known principles, the Tₘ of an oligomer compound, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligomer. For this reason, compounds that show high Tₘ (50° C. or greater) at a length of 20 bases or less are generally preferred over those requiring greater than 20 bases for high Tₘ values.

The antisense activity of the oligomer may be enhanced by using a mixture of uncharged and cationic phosphorodiamidate linkages as shown in FIGS. 2G and 2H. The total number of cationic linkages in the oligomer can vary from 1 to 10, and be interspersed throughout the oligomer. Preferably the number of charged linkages is at least 2 and no more than half the total backbone linkages, e.g., between 2-8 positively charged linkages, and preferably each charged linkages is separated along the backbone by at least one, preferably at least two uncharged linkages. The antisense activity of various oligomers can be measured in vitro by fusing the oligomer target region to the 5′ end a reporter gene (e.g. firefly luciferase) and then measuring the inhibition of translation of the fusion gene mRNA transcripts in cell free translation assays. The inhibitory properties of oligomers containing a mixture of uncharged and cationic linkages can be enhanced between, approximately, five to 100 fold in cell free translation assays.

Table 2 below shows exemplary targeting sequences, in a 5′-to-3′ orientation, that target the FMDV-A24 genotype (GenBank Acc. No. AY593768) according to the guidelines described above. The sequences listed provide a collection of targeting sequences from which targeting sequences may be selected, according to the general class rules discussed above. SEQ ID NOS:9-15 are antisense to the positive strand (mRNA) of the viral RNA. As indicated above, where the targeting sequence is designed to bind to a consensus RNA sequence, a G subunit is selected to bind to a target C/T variation. Where the base in a target sequence is either an “R” or “N,” the corresponding targeting base should be inosine (1), which is capable of forming a base duplex with any target base.

TABLE 2

Exemplary Antisense Oligomer Sequences
Targeting FMDV

Target

| | |
|---|---|
| GenBank | SEQ |
| No. | ID |

| | | | |
|---|---|---|---|
| Name | AY593768 | Sequence 5′-3′ | NO |

| | | | |
|---|---|---|---|
| 5′ + | 1-21 | AACCCTAGCGCCCCCTTTCAA | 9 |
| CRE | 569-589 | CTTAGATCGTGTTTGTACAAG | 10 |
| IRES D5 | 1015-1035 | TTAAAAGAAAGGTGCCGGCCT | 11 |
| AUG1 | 1036-1056 | GTGTTCATAAGTCCAGTGTAA | 12 |
| AUG2 | 1121-1141 | GAATTCCATCCTTCCTGTGGC | 13 |
| AUG3 | 1042-1061 | CAGTTGTGTTCATAAGTCCA | 14 |
| 3′ Slab | 8085-8105 | ACTCCTACGGCGTCGCGCGCC | 15 |

Consensus Targeting

Sequences*

| | | | |
|---|---|---|---|
| IREScons | 1009-1029 | AAGGGAAAGGNGCCGGCCTCCGGTC | 29 |
| AUG1cons | 1046-1065 | CAGTCAGTNGTNTTCATGGG | 30 |
| AUG2cons | 1059-1078 | CANAGCGATAAAACAGTCAG | 31 |
| AUG3cons | 1127-1146 | AGTGTGAATTCCATNTTNCN | 32 |

*N is Inosine

III. Antisense Oligonucleotide Analog Compounds

A. Properties

As detailed above, the antisense oligonucleotide analog compound (the term “antisense” indicates that the compound is targeted against either the virus' positive-sense strand RNA or negative-sense or minus-strand) has a base sequence target region that includes one or more of the following: 1) 50 bases surrounding the AUG start codons of viral mRNA or; 2) 30 bases at the 3′ terminus of the minus strand viral RNA. In addition, the oligomer is able to effectively target infecting viruses, when administered to a host cell, e.g. in an infected mammalian subject. This requirement is met when the oligomer compound (a) has the ability to be actively taken up by mammalian cells, and (b) once taken up, form a duplex with the target RNA with a Tₘ greater than about 45° C.

As will be described below, the ability to be taken up by cells requires that the oligomer backbone be substantially uncharged, and, preferably, that the oligomer structure is recognized as a substrate for active or facilitated transport across the cell membrane. The ability of the oligomer to form a stable duplex with the target RNA will also depend on the oligomer backbone, as well as factors noted above, the length and degree of complementarity of the antisense oligomer with respect to the target, the ratio of G:C to A:T base matches, and the positions of any mismatched bases. The ability of the antisense oligomer to resist cellular nucleases promotes survival and ultimate delivery of the agent to the cell cytoplasm.

Below are disclosed methods for testing any given, substantially uncharged backbone for its ability to meet these requirements.

B. Active or Facilitated Uptake by Cells

The antisense compound may be taken up by passive diffusion into host cells, or by facilitated or active transport across the host cell membrane if administered in free (non-complexed) form, or by an endocytotic mechanism if administered in complexed form. In the latter case, the oligonucleotide compound may be a substrate for a membrane transporter system (i.e. a membrane protein or proteins) capable of facilitating transport or actively transporting the oligomer across the cell membrane. This feature may be determined by one of a number of tests for oligomer interaction or cell uptake, as follows.

A first test assesses binding at cell surface receptors, by examining the ability of an oligomer compound to displace or be displaced by a selected charged oligomer, e.g., a phosphorothioate oligomer, on a cell surface. The cells are incubated with a given quantity of test oligomer, which is typically fluorescently labeled, at a final oligomer concentration of between about 10-300 nM. Shortly thereafter, e.g., 10-30 minutes (before significant internalization of the test oligomer can occur), the displacing compound is added, in incrementally increasing concentrations. If the test compound is able to bind to a cell surface receptor, the displacing compound will be observed to displace the test compound. If the displacing compound is shown to produce 50% displacement at a concentration of 10× the test compound concentration or less, the test compound is considered to bind at the same recognition site for the cell transport system as the displacing compound.

A second test measures cell transport, by examining the ability of the test compound to transport a labeled reporter, e.g., a fluorescence reporter, into cells. The cells are incubated in the presence of labeled test compound, added at a final concentration between about 10-300 nM. After incubation for 30-120 minutes, the cells are examined, e.g., by microscopy, for intracellular label. The presence of significant intracellular label is evidence that the test compound is transported by facilitated or active transport.

The antisense compound may also be administered in complexed form, where the complexing agent is typically a polymer, e.g., a cationic lipid, polypeptide, or non-biological cationic polymer, having an opposite charge to any net charge on the antisense compound. Methods of forming complexes, including bilayer complexes, between anionic oligonucleotides and cationic lipid or other polymer components, are well known. For example, the liposomal composition Lipofectin® (Felgner, Gadek et al. 1987), containing the cationic lipid DOTMA (N-[1-(2,3-dioleyloxy)propyl]-N,N,N-trimethylammonium chloride) and the neutral phospholipid DOPE (dioleyl phosphatidyl ethanolamine), is widely used. After administration, the complex is taken up by cells through an endocytotic mechanism, typically involving particle encapsulation in endosomal bodies.

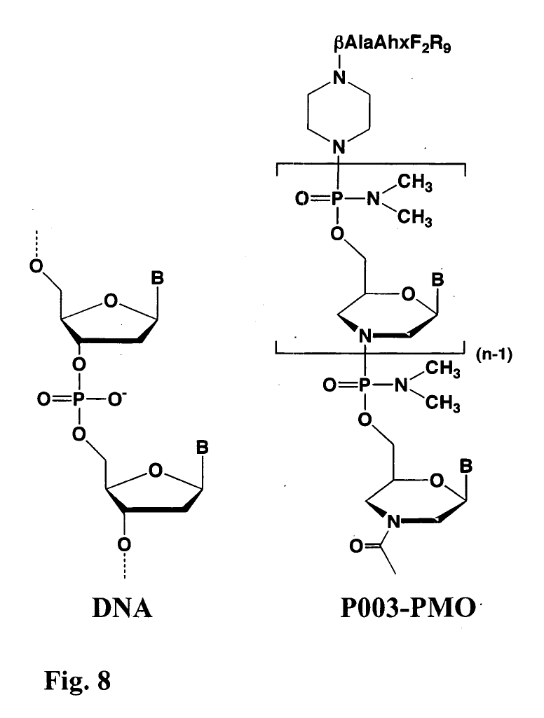

The antisense compound may also be administered in conjugated form with an arginine-rich peptide linked covalently to the 5′ or 3′ end of the antisense oligomer. The peptide is typically 8-16 amino acids and consists of a mixture of arginine, and other amino acids including phenyalanine and cysteine. The peptide may also contain non-natural amino acids such as beta-alanine and 6-aminohexanoic acid. Exemplary arginine-rich peptide are listed as SEQ ID NOS: 33-35. An example of an arginine-rich delivery peptide used to conduct experiments in support of the invention is shown in FIG. 8. The use of arginine-rich peptide-PMO conjugates can be used to enhance cellular uptake of the antisense oligomer (See, e.g. (Moulton, Nelson et al. 2004).

In some instances, liposomes may be employed to facilitate uptake of the antisense oligonucleotide into cells. (See, e.g., Williams, S. A., Leukemia 10(12):1980-1989, 1996; Lappalainen et al., Antiviral Res. 23:119, 1994; Uhlmann et al., antisense oligonucleotides: a new therapeutic principle, Chemical Reviews, Volume 90, No. 4, pages 544-584, 1990; Gregoriadis, G., Chapter 14, Liposomes, Drug Carriers in Biology and Medicine, pp. 287-341, Academic Press, 1979). Hydrogels may also be used as vehicles for antisense oligomer administration, for example, as described in WO 93/01286. Alternatively, the oligonucleotides may be administered in microspheres or microparticles. (See, e.g., Wu, G. Y. and Wu, C. H., J. Biol. Chem. 262:4429-4432, 1987). Alternatively, the use of gas-filled microbubbles complexed with the antisense oligomers can enhance delivery to target tissues, as described in U.S. Pat. No. 6,245,747.

Alternatively, and according to another aspect of the invention, the requisite properties of oligomers with any given backbone can be confirmed by a simple in vivo test, in which a labeled compound is administered to an animal, and a body fluid sample, taken from the animal several hours after the oligomer is administered, assayed for the presence of heteroduplex with target RNA. This method is detailed in subsection D below.

C. Substantial Resistance to RNaseH

Two general mechanisms have been proposed to account for inhibition of expression by antisense oligonucleotides. (See e.g., (Agrawal, Mayrand et al. 1990; Bonham, Brown et al. 1995; Boudvillain, Guerin et al. 1997). In the first, a heteroduplex formed between the oligonucleotide and the viral RNA acts as a substrate for RNaseH, leading to cleavage of the viral RNA. Oligonucleotides belonging, or proposed to belong, to this class include phosphorothioates, phosphotriesters, and phosphodiesters (unmodified “natural” oligonucleotides). Such compounds expose the viral RNA in an oligomer:RNA duplex structure to hydrolysis by RNaseH, and therefore loss of function.

A second class of oligonucleotide analogs, termed “steric blockers” or, alternatively, “RNaseH inactive” or “RNaseH resistant”, have not been observed to act as a substrate for RNaseH, and are believed to act by sterically blocking target RNA nucleocytoplasmic transport, splicing or translation. This class includes methylphosphonates (Toulme et al., 1996), morpholino oligonucleotides, peptide nucleic acids (PNA's), certain 2′-O-allyl or 2′-O-alkyl modified oligonucleotides (Bonham, 1995), and N3′→4P5′ phosphoramidates (Gee, 1998; Ding, 1996).

A test oligomer can be assayed for its RNaseH resistance by forming an RNA:oligomer duplex with the test compound, then incubating the duplex with RNaseH under a standard assay conditions, as described in Stein et al. After exposure to RNaseH, the presence or absence of intact duplex can be monitored by gel electrophoresis or mass spectrometry.

D. In Vivo Uptake

In accordance with another aspect of the invention, there is provided a simple, rapid test for confirming that a given antisense oligomer type provides the required characteristics noted above, namely, high Tₘ ability to be actively taken up by the host cells, and substantial resistance to RNaseH. This method is based on the discovery that a properly designed antisense compound will form a stable heteroduplex with the complementary portion of the viral RNA target when administered to a mammalian subject, and the heteroduplex subsequently appears in the urine (or other body fluid). Details of this method are also given in co-owned U.S. patent application Ser. No. 09/736,920, entitled “Non-Invasive Method for Detecting Target RNA” (Non-Invasive Method), the disclosure of which is incorporated herein by reference.

Briefly, a test oligomer containing a backbone to be evaluated, having a base sequence targeted against a known RNA, is injected into a mammalian subject. The antisense oligomer may be directed against any intracellular RNA, including a host RNA or the RNA of an infecting virus. Several hours (typically 8-72) after administration, the urine is assayed for the presence of the antisense-RNA heteroduplex. If heteroduplex is detected, the backbone is suitable for use in the antisense oligomers of the present invention.

The test oligomer may be labeled, e.g. by a fluorescent or a radioactive tag, to facilitate subsequent analyses, if it is appropriate for the mammalian subject. The assay can be in any suitable solid-phase or fluid format. Generally, a solid-phase assay involves first binding the heteroduplex analyte to a solid-phase support, e.g., particles or a polymer or test-strip substrate, and detecting the presence/amount of heteroduplex bound. In a fluid-phase assay, the analyte sample is typically pretreated to remove interfering sample components. If the oligomer is labeled, the presence of the heteroduplex is confirmed by detecting the label tags. For non-labeled compounds, the heteroduplex may be detected by immunoassay if in solid phase format or by mass spectroscopy or other known methods if in solution or suspension format.

When the antisense oligomer is complementary to a virus-specific region of the viral genome (such as those regions of influenza RNA, as described above) the method can be used to detect the presence of a given FMDV virus, or reduction in the amount of virus during a treatment method.

E. Exemplary Oligomer Backbones

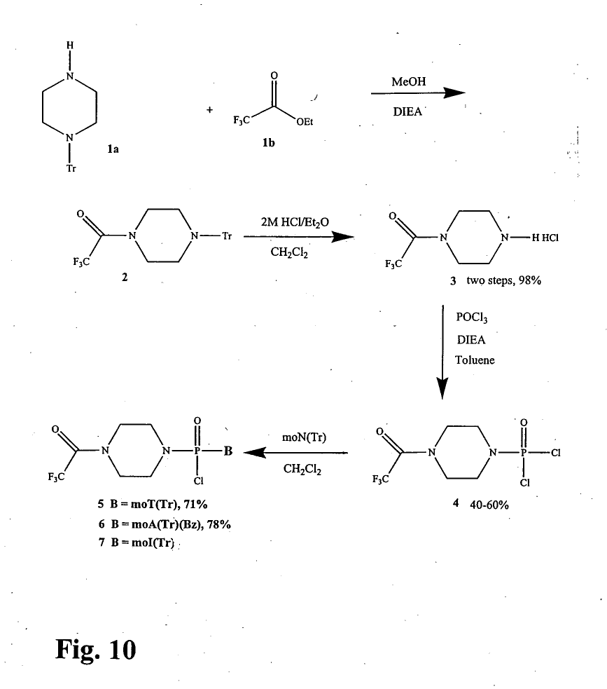

Examples of nonionic linkages that may be used in oligonucleotide analogs are shown in FIGS. 2A-2G. In these figures, B represents a purine or pyrimidine base-pairing moiety effective to bind, by base-specific hydrogen bonding, to a base in a polynucleotide, preferably selected from adenine, cytosine, guanine and uracil. Suitable backbone structures include carbonate (2A, R=O) and carbamate (2A, R=NH₂) linkages (Mertes and Coats 1969; Gait, Jones et al. 1974); alkyl phosphonate and phosphotriester linkages (2B, R=alkyl or —O-alkyl) (Lesnikowski, Jaworska et al. 1990); amide linkages (2C) (Blommers, Pieles et al. 1994); sulfone and sulfonamide linkages (2D, R₁, R₂=CH₂); and a thioformacetyl linkage (2E) (Cross, Rice et al. 1997). The latter is reported to have enhanced duplex and triplex stability with respect to phosphorothioate antisense compounds (Cross, Rice et al. 1997). Also reported are the 3′-methylene-N-methylhydroxyamino compounds of structure 2F. Also shown is a cationic linkage in FIG. 2H wherein the nitrogen pendant to the phosphate atom in the linkage of FIG. 2G is replaced with a 1-piperazino structure. The method for synthesizing the 1-piperazino group linkages is described below with respect to FIG. 10.

Peptide nucleic acids (PNAs) are analogs of DNA in which the backbone is structurally homomorphous with a deoxyribose backbone, consisting of N-(2-aminoethyl)glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm, Buchardt et al. 1993). The backbone of PNAs are formed by peptide bonds rather than phosphodiester bonds, making them well-suited for antisense applications. The backbone is uncharged, resulting in PNA/DNA or PNA/RNA duplexes which exhibit greater than normal thermal stability. PNAs are not recognized by nucleases or proteases.

A preferred oligomer structure employs morpholino-based subunits bearing base-pairing moieties, joined by uncharged linkages, as described above. Especially preferred is a substantially uncharged phosphorodiamidate-linked morpholino oligomer, such as illustrated in FIGS. 1A-1D. Morpholino oligonucleotides, including antisense oligomers, are detailed, for example, in co-owned U.S. Pat. Nos. 5,698,685, 5,217,866, 5,142,047, 5,034,506, 5,166,315, 5,185,444, 5,521,063, and 5,506,337, all of which are expressly incorporated by reference herein.

Important properties of the morpholino-based subunits include: the ability to be linked in a oligomeric form by stable, uncharged backbone linkages; the ability to support a nucleotide base (e.g. adenine, cytosine, guanine, thymidine, inosine or uracil) such that the polymer formed can hybridize with a complementary-base target nucleic acid, including target RNA, with high Tₘ, even with oligomers as short as 10-14 bases; the ability of the oligomer to be actively transported into mammalian cells; and the ability of the oligomer:RNA heteroduplex to resist RNAse degradation.

Exemplary backbone structures for antisense oligonucleotides of the invention include the β-morpholino subunit types shown in FIGS. 1A-1D, each linked by an uncharged, phosphorus-containing subunit linkage. FIG. 1A shows a phosphorus-containing linkage which forms the five atom repeating-unit backbone, where the morpholino rings are linked by a 1-atom phosphoamide linkage. FIG. 1B shows a linkage which produces a 6-atom repeating-unit backbone. In this structure, the atom Y linking the 5′ morpholino carbon to the phosphorus group may be sulfur, nitrogen, carbon or, preferably, oxygen. The X moiety pendant from the phosphorus may be fluorine, an alkyl or substituted alkyl, an alkoxy or substituted alkoxy, a thioalkoxy or substituted thioalkoxy, or unsubstituted, monosubstituted, or disubstituted nitrogen, including cyclic structures, such as morpholines or piperidines. Alkyl, alkoxy and thioalkoxy preferably include 1-6 carbon atoms. The Z moieties are sulfur or oxygen, and are preferably oxygen.

The linkages shown in FIGS. 1C and 1D are designed for 7-atom unit-length backbones. In Structure 1C, the X moiety is as in Structure 1B, and the moiety Y may be methylene, sulfur, or, preferably, oxygen. In Structure 1D, the X and Y moieties are as in Structure 1B. Particularly preferred morpholino oligonucleotides include those composed of morpholino subunit structures of the form shown in FIG. 1B, where X=NH₂ or N(CH₃)₂, Y=O, and Z=O. This preferred structure, as described, is also shown in FIG. 2G.

As noted above, the substantially uncharged oligomer may advantageously include a limited number of charged backbone linkages. One example of a cationic charged phophordiamidate linkage is shown in FIG. 2H. This linkage, in which the dimethylamino group shown in FIG. 2G is replaced a 1-piperazino group as shown in FIG. 2G, can be substituted for any linkage(s) in the oligomer. By including between two to eight such cationic linkages, and more generally, at least two and no more than about half the total number of linkages, interspersed along the backbone of the otherwise uncharged oligomer, antisense activity can be enhanced without a significant loss of specificity. The charged linkages are preferably separated in the backbone by at least 1 and preferably 2 or more uncharged linkages.

The antisense compounds can be prepared by stepwise solid-phase synthesis, employing methods detailed in the references cited above. In some cases, it may be desirable to add additional chemical moieties to the antisense compound, e.g. to enhance pharmacokinetics or to facilitate capture or detection of the compound. Such a moiety may be covalently attached, typically to a terminus of the oligomer, according to standard synthetic methods. For example, addition of a polyethyleneglycol moiety or other hydrophilic polymer, e.g., one having 10-100 monomeric subunits, may be useful in enhancing solubility. One or more charged groups, e.g., anionic charged groups such as an organic acid, may enhance cell uptake. A reporter moiety, such as fluorescein or a radiolabeled group, may be attached for purposes of detection. Alternatively, the reporter label attached to the oligomer may be a ligand, such as an antigen or biotin, capable of binding a labeled antibody or streptavidin. In selecting a moiety for attachment or modification of an antisense oligomer, it is generally of course desirable to select chemical compounds of groups that are biocompatible and likely to be tolerated by a subject without undesirable side effects.

IV. Inhibition of FMDV Replication

A. Inhibition in BHK-21 Cells:

PMO antisense compounds and the negative control PMOs (SEQ ID NOS: 16 and 17) were initially evaluated of for cytotoxicity when incubated with BHK-21 cells using a standard MTT assay. A window of PMO concentration was chosen (1-5 micromolar) between which the virus was inhibited and cell death was less than 20%.

Antisense FMDV PMOs were designed to target regions in the 5′ and 3′ untranslated regions (UTRs) of the FMDV genome as described above. PMOs that targeted regions involved in both translation (IRES 5D, AUG1, AUG2 and AUG3; SEQ ID NOS: 11-14, respectively) and replication (5′+, CRE and 3′SL; SEQ ID NOS: 9,10 and 15, respectively) were used in tissue culture experiments to determine their relative antiviral effects. The PMO targets are shown schematically in FIG. 3. All PMOs were conjugated at the 5′ terminus with an arginine-rich delivery peptide (R₉F₂Ahxβala) as shown in FIG. 4.

All PMOs evaluated to date reduced the viral titer, as measured by plaque forming unit (PFU)-to some degree as described further in Example 1. The three PMOs that targeted regions thought to be essential for translation (IRES 5D, AUG1, AUG2; SEQ ID NOS: 11-13, respectively) were the most inhibitory against the FMDV A24 Cruzeiro genotype. Table 4 below lists the virus titers express as PFU/ml of antiviral PMOs (2.5 μM) compared to negative control PMOs (DSscr) and untreated cultures.

TABLE 4

FMDV Titer Reduction in BHK-21 Cells.

Inhibition Log₁₀

| | |
|---|---|
| Treatment | (PFU/ml) relative |

| | | |
|---|---|---|
| (PMO, 2.5 uM) | to DSscr | PFU/ml |

| | | |
|---|---|---|
| Control (no treatment) | ND | 4.8 10⁷ |
| DSscr (SEQ ID NO:16) | 0 | 1.9 10⁷ |
| AUG2scr (SEQ ID NO:17) | 0.05 +/− 0.04 | 1.7 10⁷ |
| 5′ + (SEQ ID NO:9) | 0.64 +/− 0.36 | 5.7 10⁶ |
| CRE (SEQ ID NO:10) | 0.59 +/− 0.26 | 5.8 10⁶ |
| IRES D5 (SEQ ID NO:11) | 5.11 +/− 1.35 | 7.4 10² |
| AUG1 (SEQ ID NO:12) | 6.31 +/− 0.14 | 1.0 10¹ |
| AUG2 (SEQ ID NO:13) | 6.37 +/− 0.23 | 8.3 10¹ |
| 3′ SLab (SEQ ID NO:15) | 0.12 +/− 0.13 | 1.4 10⁷ |

Table 4, above, shows that PMOs targeting the IRES domain 5 (IRES D5; SEQ ID NO:11) and the AUG1 and AUG2 start-sites (SEQ ID NOS: 12 and 13, respectively) of FMDVA24 showed the strongest antiviral activity generating virus titer reductions greater than 5 log₁₀. A concomitant inhibition of viral protein and RNA synthesis was observed in a dose-dependent manner as described further in Example 1. Under similar conditions, three other compounds, targeting the 5′-terminal 21 nucleotides of the genome (5′+; SEQ ID NO:9), the cis-acting replication element (CRE; SEQ ID NO:10) and the 3′stem-loop ab (3′Slab; SEQ ID NO: 15), showed only moderate suppression (less than one log₁₀) of viral replication. No specific inhibition was seen when the PMOs at 2.5 μM were tested with Bovine Enterovirus (BEV) infection in BHK-21 cells as described in Example 1. A two Log₁₀ reduction observed for PMO 5D against BEV could be explained by partial match between this particular PMO to at least three different target sequences within the BEV genome (data not shown). Negative control PMOs consisting of a scrambled AUG2 sequence (AUG2scr; SEQ ID NO:17) and an irrelevant sequence (DSscr; SEQ ID NO:16) showed no inhibition on FMDV further demonstrating the sequence specificity of the antiviral PMOs. Treatment with 2.5 uM PMO IRES D5 reduced the titer of FMDV serotypes A, O, C and Asia 1 by over 4 log₁₀ compared to controls indicated this compound has broad specificity as predicted from the sequence homology amongst different serotypes at this target.

The results from the experiments conducted in support of the invention indicate that PMOs targeting those areas involved in viral translation have great potential as antiviral agents for treating FMDV infection as well as tools for molecular studies of viral translation and replication. More importantly PMO targeting IRES domain 5 (IRES 5D; SEQ ID NO:11) has proved to be a selective and potent inhibitor of the replication of the 6 tested pathogenic FMDV serotypes (A, O, C SAT1, SAT2 and Asia1) at concentrations which did not alter normal cell viability.

V. Treatment Method

The antisense compounds detailed above are useful in inhibiting an FMDV infection in a mammalian subject. In this method the oligonucleotide antisense compound of the invention is administered to a mammalian subject, e.g., domestic, cloven-hoofed animal, infected with the virus, in a suitable pharmaceutical carrier. The treatment method is intended to reduce the viral count in the infected animal sufficiently to arrest the infection and allow for eventual immunity and cure.

A. Identification of the Infective Agent

The specific FMDV strain causing the infection can be determined by methods known in the art, e.g. serological or cultural methods, or by methods employing the antisense oligomers of the present invention. Identification of the specific viral strain is not necessary if an antisense oligomer with broad specificity as exemplified by the IRES 5D PMO (SEQ ID NO:11) is employed.

Serological identification employs a viral sample or culture isolated from a biological specimen, e.g., stool, urine, cerebrospinal fluid, blood, etc., of the subject. Immunoassay for the detection of virus is generally carried out by methods routinely employed by those of skill in the art, e.g., ELISA or Western blot In addition, monoclonal antibodies specific to particular viral strains or species are often commercially available.

Another method for identifying the FMDV serotype employs one or more antisense oligomers targeting specific viral strains. In this method, (a) the oligomer(s) are administered to the subject; (b) at a selected time after said administering, a body fluid sample is obtained from the subject; and (c) the sample is assayed for the presence of a nuclease-resistant heteroduplex comprising the antisense oligomer and a complementary portion of the viral genome. Steps (a)-(c) are carried for at least one such oligomer, or as many as is necessary to identify the virus or family of viruses. Oligomers can be administered and assayed sequentially or, more conveniently, concurrently. The viral strain is identified based on the presence (or absence) of a heteroduplex comprising the antisense oligomer and a complementary portion of the viral genome of the given known virus or family of viruses.

Preferably, a first group of oligomers, targeting broad families, is utilized first, followed by selected oligomers complementary to specific genera and/or species and/or strains within the broad family/genus thereby identified. This second group of oligomers includes targeting sequences directed to specific genera and/or species and/or strains within a broad family/genus. Several different second oligomer collections, i.e. one for each broad virus family/genus tested in the first stage, are generally provided Sequences are selected which are (i) specific for the individual genus/species/strains being tested and (ii) not found in humans.

B. Administration of the Antisense Oligomer

Effective delivery of the antisense oligomer to the target nucleic acid is an important aspect of treatment. In accordance with the invention, routes of antisense oligomer delivery include, but are not limited to, various systemic routes, including oral and parenteral routes, e.g., intravenous, subcutaneous, intraperitoneal, and intramuscular, as well as inhalation, transdermal and topical delivery. The appropriate route may be determined by one of skill in the art, as appropriate to the condition of the subject under treatment. For example, an appropriate route for delivery of an antisense oligomer in the treatment of a viral infection of the skin is topical delivery, while delivery of an antisense oligomer for the treatment of a viral respiratory infection is by inhalation. The oligomer may also be delivered directly to the site of viral infection, or to the bloodstream.

The antisense oligomer may be administered in any convenient vehicle which is physiologically acceptable. Such a composition may include any of a variety of standard pharmaceutically accepted carriers employed by those of ordinary skill in the art. Examples include, but are not limited to, saline, phosphate buffered saline (PBS), water, aqueous ethanol, emulsions, such as oil/water emulsions or triglyceride emulsions, tablets and capsules. The choice of suitable physiologically acceptable carrier will vary dependent upon the chosen mode of administration.

In some instances, liposomes may be employed to facilitate uptake of the antisense oligonucleotide into cells. (See, e.g., Williams, S. A., Leukemia 10(12):1980-1989, 1996; Lappalainen et al., Antiviral Res. 23:119, 1994; Uhlmann et al., antisense oligonucleotides: a new therapeutic principle, Chemical Reviews, Volume 90, No. 4, pages 544-584, 1990; Gregoriadis, G., Chapter 14, Liposomes, Drug Carriers in Biology and Medicine, pp. 287-341, Academic Press, 1979). Hydrogels may also be used as vehicles for antisense oligomer administration, for example, as described in WO 93/01286. Alternatively, the oligonucleotides may be administered in microspheres or microparticles. (See, e.g., Wu, G. Y. and Wu, C. H., J. Biol. Chem. 262:4429-4432, 1987). Alternatively, the use of gas-filled microbubbles complexed with the antisense oligomers can enhance delivery to target tissues, as described in U.S. Pat. No. 6,245,747.

Sustained release compositions may also be used. These may include semipermeable polymeric matrices in the form of shaped articles such as films or microcapsules.

The antisense compound is generally administered in an amount and manner effective to result in a peak blood concentration of at least 200-400 nM antisense oligomer. Typically, one or more doses of antisense oligomer are administered, generally at regular intervals, for a period of about one to two weeks. Preferred doses for oral administration are from about 5-500 mg oligomer or oligomer cocktail per 70 kg individual. In some cases, doses of greater than 500 mg oligomer/subject may be necessary. For i.v. or i.p. administration, preferred doses are from about 1-250 mg oligomer or oligomer cocktail per 70 kg body weight. The antisense oligomer may be administered at regular intervals for a short time period, e.g., daily for two weeks or less. However, in some cases the oligomer is administered intermittently over a longer period of time. Administration may be followed by, or concurrent with, administration of an antibiotic or other therapeutic treatment. The treatment regimen may be adjusted (dose, frequency, route, etc.) as indicated, based on the results of immunoassays, other biochemical tests and physiological examination of the subject under treatment.

VI. Preparation Of Morpholino Oligomers Having Cationic Linkages

A schematic of a synthetic pathway that can be used to make morpholino subunits containing a (1-piperazino) phosphinylideneoxy linkage is shown in FIG. 10; further experimental detail for a representative synthesis is provided in Materials and Methods, below. As shown in the Figure, reaction of piperazine and trityl chloride gave trityl piperazine (1a), which was isolated as the succinate salt. Reaction with ethyl trifluoroacetate (1b) in the presence of a weak base (such as diisopropylethylamine or DIEA) provided 1-trifluoroacetyl-4-trityl piperazine (2), which was immediately reacted with HCl to provide the salt (3) in good yield. Introduction of the dichlorophosphoryl moiety was performed with phosphorus oxychloride in toluene.

The acid chloride (4) is reacted with morpholino subunits (moN), which may be prepared as described in U.S. Pat. No. 5,185,444 or in Summerton and Weller, 1997 (cited above), to provide the activated subunits (5,6,7). Suitable protecting groups are used for the nucleoside bases, where necessary, for example, benzoyl for adenine and cytosine, isobutyryl for guanine, and pivaloylmethyl for inosine. The subunits containing the (1-piperazino) phosphinylideneoxy linkage can be incorporated into the existing PMO synthesis protocol, as described, for example in Summerton and Weller (1997), without modification.

### EXAMPLES OF THE INVENTION

Materials and Methods

All peptides were custom synthesized by Global Peptide Services (Ft. Collins, Colo.) or at AVI BioPharma (Corvallis, Oreg.) and purified to >90% purity (see Example 2 below). PMOs were synthesized at AVI BioPharma in accordance with known methods, as described, for example, in ((Summerton and Weller 1997) and U.S. Pat. No. 5,185,444.

PMO oligomers were conjugated at the 5′ end with an arginine-rich peptide (R₉F₂AhxβAla-5′-PMO, SEQ ID NO:18) to enhance cellular uptake as described (U.S. Patent Application 60/466,703 and (Moulton, Nelson et al. 2004). Beta-Alanine (βAla) and 6-aminohexanoic acid (Ahx) are non-natural amino acids.

Oligomer Synthesis

Preparation of N-trityl piperazine, succinate salt (1a): To a cooled solution of piperazine (10 eq) in toluene/methanol (5:1 toluene/methanol (v:v); 5 mL/g piperazine) was added slowly a solution of trityl chloride (1.0 eq) in toluene (5 mL/g trityl chloride). Upon reaction completion (1-2 hours), this solution was washed 4× with water. To the resulting organic solution was added an aqueous solution of succinic acid (1.1 eq; 13 mL water/g succinic acid). This mixture was stirred for 90 minutes, and the solid product was collected by filtration. The crude solid was purified by two reslurries in acetone. Yield=70%.

Preparation of 1-trifluoroacetyl-4-trityl piperazine (2): To a slurry of 1a in methanol (10 mL/g 1a) was added diisopropylethylamine (2.1 eq) and ethyl trifluoroacetate (1.2 eq). After overnight stirring, the organic mixture was distilled to dryness. The resulting oil was dissolved in DCM (10 mL/g 1a) and washed 3× with 5% NaCl/H₂O. This solution was dried over Na₂SO₄, then concentrated to give a white foam. Yield=100%. ¹⁹F NMR (CDCl₃) δ −68.7 (s).

Preparation of N-trifluoroacetyl piperazine, HCl salt (3): To a solution of 2 in DCM (10 mL/g 2) was added dropwise a solution of 2.0 M HCl/Et₂O (2.1 eq). The reaction mixture was stirred for 4 hours, and the product was collected by filtration. The filter cake was washed 3× with DCM. The solid was dried at 40° C. in a vacuum oven for 24 hours. Yield=95%. ¹⁹F NMR (CDCl₃) δ −68.2 (s); melting point=154-156° C.

Preparation of Activating Agent (4): To a cooled mixture of 3 (1.0 eq) and diisopropylethylamine (4.0 eq) in toluene (20 mL/g 3) was added slowly a solution of POCl₃ (1.1 eq) in toluene (20 mL/g 3). The reaction mixture was stirred in an ice bath for 4 hours. The reaction mixture was diluted with additional toluene (20 mL/g 3) and washed twice with 1 M KH₂PO₄ and once with 5% NaCl/H₂O. This solution was dried over Na₂SO₄ and distilled to an oil, which was then purified by silica gel chromatography (10% ethyl acetate/heptane as eluent). Yield=50%. ¹⁹F NMR (CDCl₃) δ −68.85 (s); ³¹P NMR (CDCl₃) δ 15.4 (s).

Preparation of Activated Subunits (5, 6). To a cooled solution of 4 (1.2 eq) in DCM (10 mL/g 4) were added successively 2,6-lutidine (2.0 eq), N-methylimidazole (0.3 μeq), and tritylated, base-protected (where necessary) morpholino subunit (1.0 eq). The solution was allowed to warm to room temperature. After 6 hours, the solution was washed with 1 M citric acid (pH 3). The organic layer was dried over Na₂SO₄, and the solvents were removed. The crude product was purified by silica gel chromatography (gradient of ethyl acetate/heptane). Yield=60-70%. Data for 5: ¹⁹F NMR (CDCl₃) δ −68.823 (s), −68.832 (s); ³¹P NMR (CDCl₃) δ 13.167 (s), 13.038 (s). Data for 6: ¹⁹F NMR (CDCl₃) δ −68.826 (s), −68.833 (s); ³¹P NMR (CDCl₃) δ 13.322 (s), 13.101 (s).

### Example 1

### Inhibition of FMDV Replication in Tissue Culture

The antiviral activity of FMDV-specific PMOs was determined by the virus titer reduction on infected BHK-21 cells. The test is performed on BHK-21 cell monolayers (12-well plates) with the pretreatment of each cell monolayer with a particular PMO generally from 1 to 5 uM. Following three hours of treatment with the antiviral compound, the media is removed and the virus is added to the cells and the incubation proceeds for 1 h at 37° C. Following adsorption, the unbound virus is removed and the media is replaced by fresh PMO at the same concentration used during pretreatment, and the infection is allowed to proceed for 24 h at 37° C. Virus yield is determined by plaque assay following three freeze-thaw cycles of infected/treated BHK-21 cells. Cytotoxicity is typically evaluated by determining live cells under increasing drug concentration using an assay well known in the art (MTT Assay). A window of drug concentrations was selected between which viral replication was inhibited without killing the cells (no more than 20% of cell-death).

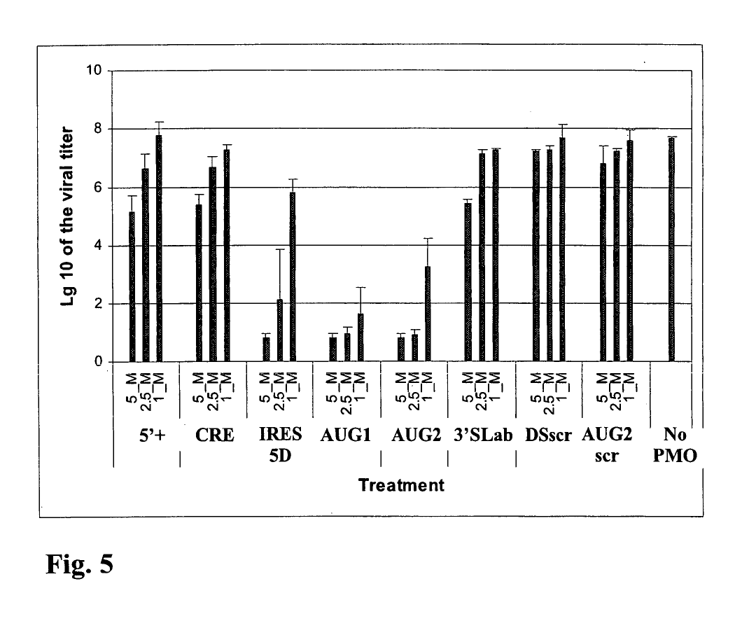

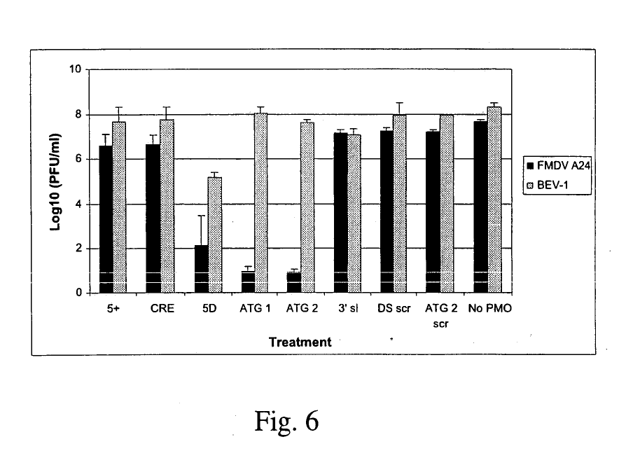

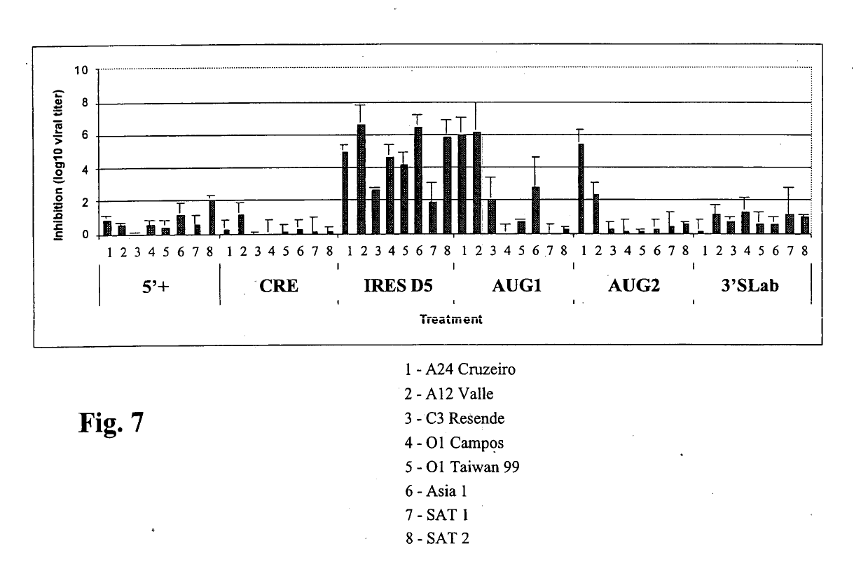

Compounds whose complementary sequences match regions in the 5′ and 3′nontranslated regions in the FMDV-genome were designed to target a range of functions thought to be involved in either viral translation (IRES 5D, AUG1, and AUG2), and replication (5+, CRE, and 3′SLab) shown schematically in FIG. 4. FIG. 5 shows that PMOs, targeting IRES D5 and both the AUG1 and AUG2 translational initiation sites (SEQ ID NOS: 11-13, respectively) showed the strongest antiviral activity generating a reduction in viral titer of greater than 5 log₁₀ with a concomitant inhibition of viral protein and RNA synthesis in a dose-dependent manner. Under similar conditions, three other compounds, targeting the 5′-terminal 21 nucleotides of the genome (5′+; SEQ ID NO:9), the cis-acting replication element (CRE; SEQ ID NO:10) and 3′stem-loop ab (3′SLab; SEQ ID NO:15), showed only moderate suppression (1.5 to 2 log₁₀) of viral replication. No specific inhibition was seen when the PMOs were tested against Bovine Enterovirus (BEV) infection in BHK-21 (FIG. 6). A scrambled-sequence (scrAUG2) PMO-control showed no inhibition on FMDV, further demonstrating genome-sequence specificity (FIG. 5). More importantly treatment with 2.5 uM PMO IRES domain 5 reduced the titer of FMDV serotypes A, O, C and Asia 1 by over 4 log₁₀ compared to controls as shown in FIG. 7.

The results demonstrate that PMOs targeting those areas involved in viral translation (IRES D5, AUG 1, and AUG 2) have great potential as antiviral agents for treating FMDV infection as well as to be considered good tools for molecular studies of viral translation and replication More importantly PMO IRES domain 5 (IRES D5; SEQ ID NO:11) has proved to be a selective and potent inhibitor of the replication of the 6 tested pathogenic FMDV serotypes (A, O, C SAT 1, SAT2 and Asia1) at concentrations, which did not alter normal cell viability (FIGS. 5 and 7).

The above example describes the design, development and testing of a new generation of FMDV-specific phosphorodiamidate morpholino-oligomer (PMO)-based antiviral drugs using the rational design of the viral targets. The antiviral compounds rapidly induce inhibition of the replication of multiple FMDV serotypes.

Sequence Listing Table

SEQ
ID

| | | |
|---|---|---|
| Name | Target Sequences (5′-3′) | NO |

| | | |
|---|---|---|
| 5′ + | TTGAAAGGGGGCGCTAGGGTT | 1 |
| CRE | CTTGTACAAACACGATCTAAG | 2 |
| IRES D5 | AGGCCGGCACCTTTCTTTTAA | 3 |
| AUG1 | TTACACTGGACTTATGAACAC | 4 |
| AUG2 | GCCACAGGAAGGATGGAATTC | 5 |
| 3′ Slab | GGCGCGCGACGCCGTAGGAGT | 6 |
| AUG1-2 | AGGCCGGCACCTTTCTTTTAATTACACTGGACTTAT | 7 |
| | GAACACAACTGATTGTTTTATCGCTTTGGTACACGC | |
| | TATCAGAGAGATCAGAGCATTTTTCCTACCACGAGC | |
| | CACAGGAAGGATGGAATTCACACTGCACAACGGTGA | |
| 5′ UTR | TTGAAAGGGGGCGCTAGGGTTTCACCCCTAGCATGC | 8 |
| | CAACGACAGTCCCCGCGTTGCACTCCACACTCACGT | |
| | TGTGCGTGCGCGGAGCTCGATGGACTATCGTTCACC | |
| | CACCTACAGCTGGACTCACGGCACCGTGTGGCCACT | |
| | TGGCTGGATTGTGCGGACGAACACCGCTTGCGCTTC | |
| | TCGCGTGACCGGTTAGTACTCTCACCACCTTCCGCC | |
| | CACTTGGTTGTTAGCGCTGTCTTGGGCACTCCTGTT | |
| | GGGGGCCGTTCGACGCTCCGCGAGTTTCCCCGCACG | |
| | GCAACTACGGTGATGGGGCCGTACCGCGCGGGCTGA | |
| | TCGCCTGGTGTGCTTCGGCTGTCACCCGAAGCCCGC | |
| | CTTTCACCCCCCCCCCCCTAAGTTTTACCGTCGTTC | |
| | CCGACGTAAAGGGATGTAACCACAAGCTTACTACCG | |
| | CCTTTCCCGGCGTTAAAGGGATGTAACCACAAGACT | |
| | TACCTTCACCCGGAAGTAAAACGGCAACTTCACACA | |
| | GTTTTGCCCGTTTTCATGAGAAATGGGACGTCTGCG | |
| | CACGAAACGCGCCGTCGCTTGAGGAGGACTTGTACA | |
| | AACACGATCTAAGCAGGTTTCCCCAACTGACACAAA | |
| | CCGTGCAATTTGAAACTCCGCCTGGGCTTTCCAGGT | |
| | CTAGAGGGGTGACACTTTGTACTGTGTTTGACTCCA | |
| | CGTTCGATCCACTGGCGAGTGTTAGTAACAACACTG | |
| | CTGCTTCGTAGCGGAGCATGACGGCCGTGGGACCCC | |
| | CCCCTTGGTAACAAGGACCCACGGGGCCAAAAGCCA | |
| | CGTCCGAATGGACCCGTCATGTGTGCAAACCCAGCA | |
| | CAGTAGCTTTGTTGTGAAACTCACTTTAAAGTGACA | |
| | TTGATACTGGTACTCAAGCACTGGTGACAGGCTAAG | |
| | GATGCCCTTCAGGTACCCCGAGGTAACACGTGACAC | |
| | TCGGGATCTGAGAAGGGGACCGGGGCTTCTATAAAA | |
| | GCGCCCGGTTTAAAAAGCTTCTATGTCTGAATAGGT | |
| | GACCGGAGGCCGGCACCTTTCTTTTAATTACACTGG | |
| | ACTT | |

Oligomer Targeting Sequences

(5′-3′)

| | | |
|---|---|---|
| 5′ + | AACCCTAGCGCCCCCTTTCAA | 9 |
| CRE | CTTAGATCGTGTTTGTACAAG | 10 |
| IRES D5 | TTAAAAGAAAGGTGCCGGCCT | 11 |
| AUG1 | GTGTTCATAAGTCCAGTGTAA | 12 |
| AUG2 | GAATTCCATCCTTCCTGTGGC | 13 |
| AUG3 | CAGTTGTGTTCATAAGTCCA | 14 |
| 3′ Slab | ACTCCTACGGCGTCGCGCGCC | 15 |
| AUG2scr | CTCAGCTGTCGTCAGTCTACT | 16 |
| DSscr | AGTCTCGACTTGCTACCTCA | 17 |

GenBank

| | |
|---|---|
| Acc. No. | SEROTYPE SEQUENCES FOR FMDV |

| | | |
|---|---|---|
| NC_011450.1 | AGGCCGGCACCTTTCTCTACAATCACTGATACTATG | 18 |
| | AACACAACTAATTGTTTTATCGCTTTGGTATACCTT | |
| | ATCAGAGAGATTAAGACACTTTTCCGTTCAAGAACT | |
| | ACAGGAAAGATGGAATTCACACTGCATAACGGTGA | |
| NC_002554.1 | AGGTCGGCACCTTTCCTTTACAATTAATGACCCTAT | 19 |
| | GAATACAACTGACTGTTTTATCGCTGTGGTAAACGC | |
| | CATCAAAGAGGTAATAGCACTTTTCCTATCACGGAC | |
| | TGCAGGAAAAATGGAATTCACGCTACACGACGGCGA | |
| NC_004915.1 | AGGCCGGCGCCTTTCCTTTGACCACTACTGTTTACA | 20 |
| | TGAACATGACCGACTGCTTTATCGCTTTGTTGTACG | |
| | CCATCAGGGAGATCAAAGCACGACTTCTTCTACGGA | |
| | CACAAGAGAAAATGGAATTCACACTCTGCAACGGTG | |
| | A | |
| NC_004004.1 | AGGCCGGCGCCTTTCCATTACCCACTACTAAATCCA | 21 |
| | TGAATACGACTGACTGTTTTATCGCTCTGCTATACG | |
| | CTCTCAGAGAGATCAAAGCACTGTTTCTGTCACGAA | |
| | CACAAGGGAAGATGGAATTCACACTTTACAACGGTG | |
| | A | |
| NC_003992.2 | AGGCCGGCACCTTTTCCTTTACCCACAACTTACTTT | 22 |
| | ATGAATACGACTGACTGTTTTATCGCTTTGGTACAG | |
| | GCTATCAGAGAGATCAAACTTTTGTTCAAAGGAATA | |
| | CGAAAGATGGAGTTCACACTGTACAACGGTGA | |
| NC_011452.1 | AGGCCGGCACCTTTTCCTTTTATCCAACACATTTTA | 23 |
| | TGAAGACAACTGACTGTTTTGACGTTTTGCTCGAGA | |
| | TCTTTCACAGGTTCCGACACACGTTCAAGACAGACA | |
| | GGAAGATGGAATTCACACTCTACAACGGTGA | |
| NC_011451.1 | AGGCCGGCACCTTTTCCTATTTAAACCTTGATTTTA | 24 |
| | TGAAGACAACTGACTGTTTCAACGTTTTGCTCGAGA | |
| | TCCTTCACAGGTTCAGACACACATTCAAGATAAATA | |
| | GAGAGATGGAATTCACACTCTACAACGGAGA | |

CONSENSUS SEQUENCE*

| | |
|---|---|
| GACCGGAGGCCGGCRCCTTTCCCTTAATTACACTGG | 25 |
| ACCCATGAANACRACTGACTGTTTTATCGCTNTGGT | |
| ACACGCTATCAGAGAGATCAGAGCATTTTTCCTACC | |
| ACGAGCCACARGRAARATGGAATTCACACTGCACAA | |
| CGGTGA | |

CONSENSUS TARGET SEQUENCES*

| | |
|---|---|
| GACCGGAGGCCGGCRCCTTTCCCTT | 26 |
| CCCATGAANACRACTGACTGTTTTATCGCTNTG | 27 |
| RGRAARATGGAATTCACACT | 28 |

CONSENSUS TARGETING SEQUENCES**

| | |
|---|---|
| AAGGGAAAGGNGCCGGCCTCCGGTC | 29 |
| CAGTCAGTNGTNTTCATGGG | 30 |
| CANAGCGATAAAACAGTCAG | 31 |
| AGTGTGAATTCCATNTTNCN | 32 |

PEPTIDE CONJUGATES***

| | |
|---|---|
| P003 | NH₂-RRRRRRRRRFFAhxβAla-COOH |
| P007 | NH₂-(RAhxR)₄AhxβAla-COOH |
| P008 | NH₂-(RAhx)₈βAla-COOH |

*N is G, A, C or T and R is Purine = G or A
**N is Inosine
***Ahx denotes 6-aminohexanoic acid and βAla denotes beta alanine.

It is claimed:

1. An antiviral antisense composition for inhibiting replication within a host cell of foot-and-mouth disease virus (FMDV), comprising an oligonucleotide compound characterized by:
(i) a nuclease-resistant backbone,
(ii) capable of uptake by mammalian host cells,
(iii) containing between 12-40 nucleotide bases,
(iv) having a targeting sequence that is complementary to a target sequence composed of at least 12 contiguous bases within the positive-strand FMDV RNA sequence defined by SEQ ID NO:25;
(v) an ability to form with the RNA target sequence, a heteroduplex structure (a) composed of the target region of the positive sense strand of the virus and the oligonucleotide compound, and (b) characterized by a Tm of dissociation of at least 45° C.; and
(vi) an ability, at a concentration of 2.5 μM, to reduce the viral titre in cultured BHK-21 cells infected with 0.5 PFU/cell of A24 Cruzeiro strain of FMDV, at least 4 orders of magnitude.

2. The composition of claim 1, wherein said compound is composed of morpholino subunits linked by uncharged, phosphorus-containing intersubunit linkages, joining a morpholino nitrogen of one subunit to a 5′ exocyclic carbon of an adjacent subunit.

3. The composition of claim 2, wherein said morpholino subunits are joined by phosphorodiamidate linkages, in accordance with the structure: 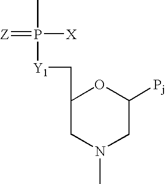
where Y₁=O, Z=O, Pj is a purine or pyrimidine base-pairing moiety effective to bind, by base-specific hydrogen bonding, to a base in a polynucleotide, and X is alkyl, alkoxy, thioalkoxy, or an alkyl amino of the form wherein NR₂, where each R is independently hydrogen or methyl.

4. The composition of claim 2, in which at least 2 and no more than half of the total number of intersubunit linkages are positively charged at physiological pH.

5. The composition of claim 4, wherein said morpholino subunits are joined by phosphorodiamidate linkages, in accordance with the structure: 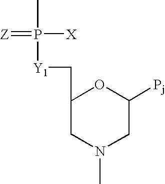
where Y₁=O, Z=O, Pj is a purine or pyrimidine base-pairing moiety effective to bind, by base-specific hydrogen bonding, to a base in a polynucleotide, and X for the uncharged linkages is alkyl, alkoxy, thioalkoxy, or an alkyl amino of the form wherein NR₂, where each R is independently hydrogen or methyl, and for the positively charged linkages, X is 1-piperazine.

6. The composition of claim 1, wherein said compound is a covalent conjugate of an oligonucleotide analog moiety capable of forming such a heteroduplex structure with the positive or negative sense strand of the virus, and an arginine-rich polypeptide effective to enhance the uptake of the compound into host cells.

7. The composition of claim 1, wherein the arginine-rich polypeptide has a sequence selected from the group consisting of SEQ ID NOS: 33-35.

8. The composition of claim 1, wherein said compound has a sequence effective to target at least 12 contiguous bases of a sequence selected from the group consisting of SEQ ID NOS: 26-28.

9. The composition of claim 8, wherein the antisense compound includes at least 15 contiguous bases of a sequences selected from the group consisting of SEQ ID NOS; 29-32.

10. The composition of claim 8, wherein the antisense compound includes a sequence selected from the group consisting of SEQ ID NOS; 29-32.

11. The composition of claim 8, wherein the antisense compound includes a sequence selected from the group consisting of SEQ ID NOS: 11-13.

12. A method of treating a FMDV infection in a veterinary animal, comprising administering to the animal, a therapeutically effective amount of an oligonucleotide analog compound characterized by:
(i) a nuclease-resistant backbone,
(ii) capable of uptake by mammalian host cells,
(iii) containing between 15-40 nucleotide bases,
(iv) having a targeting sequence that is complementary to a target sequence composed of at least 12 contiguous bases within the positive-strand FMDV RNA sequence defined by SEQ ID NO:25;
(v) an ability to form with the RNA target sequence, a heteroduplex structure (a) composed of the target region of the positive sense strand of the virus and the oligonucleotide compound, and (b) characterized by a Tm of dissociation of at least 45° C.; and
(vi) an ability, at a concentration of 2.5 μM, to reduce the viral titre in cultured BHK-21 cells infected with 0.5 PFU/cell of A24 Cruzeiro strain of FMDV, at least 4 orders of magnitude.

13. The method of claim 12, wherein the compound administered is composed of morpholino subunits linked by uncharged, phosphorus-containing intersubunit linkages, joining a morpholino nitrogen of one subunit to a 5′ exocyclic carbon of an adjacent subunit.

14. The method of claim 13, wherein said morpholino subunits are joined by phosphorodiamidate linkages, in accordance with the structure: 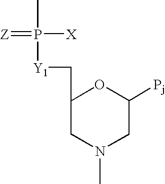
where Y₁=O, Z=O, Pj is a purine or pyrimidine base-pairing moiety effective to bind, by base-specific hydrogen bonding, to a base in a polynucleotide, and X is alkyl, alkoxy, thioalkoxy, or an alkyl amino of the form wherein NR₂, where each R is independently hydrogen or methyl.

15. The method of claim 13, in which at least 2 and no more than half of the total number of intersubunit linkages are positively charged at physiological pH.

16. The method of claim 15, wherein said morpholino subunits are joined by phosphorodiamidate linkages, in accordance with the structure: 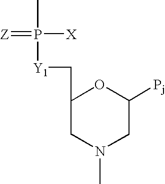
where Y₁=O, Z=O, Pj is a purine or pyrimidine base-pairing moiety effective to bind, by base-specific hydrogen bonding, to a base in a polynucleotide, and X for the uncharged linkages is alkyl, alkoxy, thioalkoxy, or an alkyl amino of the form wherein NR₂, where each R is independently hydrogen or methyl, and for the positively charged linkages, X is 1-piperazine.

17. The method of claim 12, wherein the compound administered is a covalent conjugate of an oligonucleotide analog moiety capable of forming such a heteroduplex structure with the positive or negative sense strand of the virus, and an arginine-rich polypeptide effective to enhance the uptake of the compound into host cells.

18. The method of claim 17, wherein the arginine-rich polypeptide has a sequence selected from the group consisting of SEQ ID NOS: 33-35.

19. The method of claim 18, wherein said compound has a sequence effective to target at least 12 contiguous bases of a sequence selected from the group consisting of SEQ ID NOS: 26-28.

20. The method of claim 19, wherein the antisense compound includes at least 15 contiguous bases of a sequences selected from the group consisting of SEQ ID NOS; 29-32.

21. The method of claim 19, wherein the antisense compound includes a sequence selected from the group consisting of SEQ ID NOS; 29-32.

22. The method of claim 19, wherein the antisense compound includes a sequence selected from the group consisting of SEQ ID NOS: 11-13.


SEQUENCE LISTING (35 sequences)

SEQ ID NO: 1 - DNA, 21 bp, Foot-and-mouth disease virus A
ttgaaagggggcgctagggtt

SEQ ID NO: 2 - DNA, 21 bp, Foot-and-mouth disease virus A
cttgtacaaacacgatctaag

SEQ ID NO: 3 - DNA, 21 bp, Foot-and-mouth disease virus A
aggccggcacctttcttttaa

SEQ ID NO: 4 - DNA, 21 bp, Foot-and-mouth disease virus A
ttacactggacttatgaacac

SEQ ID NO: 5 - DNA, 21 bp, Foot-and-mouth disease virus A
gccacaggaaggatggaattc

SEQ ID NO: 6 - DNA, 21 bp, Foot-and-mouth disease virus A
ggcgcgcgacgccgtaggagt

SEQ ID NO: 7 - DNA, 144 bp, Foot-and-mouth disease virus A
aggccggcacctttcttttaattacactggacttatgaacacaactgattgttttatcgctttggtacacgctatcagagagatcagagcatttttcctaccacgagccacaggaaggatggaattcacactgcacaacggtga

SEQ ID NO: 8 - DNA, 1048 bp, Foot-and-mouth disease virus A
ttgaaagggggcgctagggtttcacccctagcatgccaacgacagtccccgcgttgcactccacactcacgttgtgcgtgcgcggagctcgatggactatcgttcacccacctacagctggactcacggcaccgtgtggccacttggctggattgtgcggacgaacaccgcttgcgcttctcgcgtgaccggttagtactctcaccaccttccgcccacttggttgttagcgctgtcttgggcactcctgttgggggccgttcgacgctccgcgagtttccccgcacggcaactacggtgatggggccgtaccgcgcgggctgatcgcctggtgtgcttcggctgtcacccgaagcccgcctttcacccccccccccctaagttttaccgtcgttcccgacgtaaagggatgtaaccacaagcttactaccgcctttcccggcgttaaagggatgtaaccacaagacttaccttcacccggaagtaaaacggcaacttcacacagttttgcccgttttcatgagaaatgggacgtctgcgcacgaaacgcgccgtcgcttgaggaggacttgtacaaacacgatctaagcaggtttccccaactgacacaaaccgtgcaatttgaaactccgcctgggctttccaggtctagaggggtgacactttgtactgtgtttgactccacgttcgatccactggcgagtgttagtaacaacactgctgcttcgtagcggagcatgacggccgtgggaccccccccttggtaacaaggacccacggggccaaaagccacgtccgaatggacccgtcatgtgtgcaaacccagcacagtagctttgttgtgaaactcactttaaagtgacattgatactggtactcaagcactggtgacaggctaaggatgcccttcaggtaccccgaggtaacacgtgacactcgggatctgagaaggggaccggggcttctataaaagcgcccggtttaaaaagcttctatgtctgaataggtgaccggaggccggcacctttcttttaattacactggactt

SEQ ID NO: 9 - DNA, 21 bp, Artificial sequence
  Synthetic oligomer
aaccctagcgccccctttcaa

SEQ ID NO: 10 - DNA, 21 bp, Artificial sequence
  Synthetic oligomer
cttagatcgtgtttgtacaag

SEQ ID NO: 11 - DNA, 21 bp, Artificial sequence
  Synthetic oligomer
ttaaaagaaaggtgccggcct

SEQ ID NO: 12 - DNA, 21 bp, Artificial sequence
  Synthetic oligomer
gtgttcataagtccagtgtaa

SEQ ID NO: 13 - DNA, 21 bp, Artificial sequence
  Synthetic oligomer
gaattccatccttcctgtggc

SEQ ID NO: 14 - DNA, 20 bp, Artificial sequence
  Synthetic oligomer
cagttgtgttcataagtcca

SEQ ID NO: 15 - DNA, 21 bp, Artificial sequence
  Synthetic oligomer
actcctacggcgtcgcgcgcc

SEQ ID NO: 16 - DNA, 21 bp, Artificial sequence
  Synthetic oligomer
ctcagctgtcgtcagtctact

SEQ ID NO: 17 - DNA, 20 bp, Artificial sequence
  Synthetic oligomer
agtctcgacttgctacctca

SEQ ID NO: 18 - DNA, 143 bp, Foot-and-mouth disease virus A
aggccggcacctttctctacaatcactgatactatgaacacaactaattgttttatcgctttggtataccttatcagagagattaagacacttttccgttcaagaactacaggaaagatggaattcacactgcataacggtga

SEQ ID NO: 19 - DNA, 144 bp, Foot-and-mouth disease virus C
aggtcggcacctttcctttacaattaatgaccctatgaatacaactgactgttttatcgctgtggtaaacgccatcaaagaggtaatagcacttttcctatcacggactgcaggaaaaatggaattcacgctacacgacggcga

SEQ ID NO: 20 - DNA, 145 bp, Foot-and-mouth disease virus Asia 1
aggccggcgcctttcctttgaccactactgtttacatgaacatgaccgactgctttatcgctttgttgtacgccatcagggagatcaaagcacgacttcttctacggacacaagagaaaatggaattcacactctgcaacggtga

SEQ ID NO: 21 - DNA, 145 bp, Foot-and-mouth disease virus O
aggccggcgcctttccattacccactactaaatccatgaatacgactgactgttttatcgctctgctatacgctctcagagagatcaaagcactgtttctgtcacgaacacaagggaagatggaattcacactttacaacggtga

SEQ ID NO: 22 - DNA, 140 bp, Foot-and-mouth disease virus SAT 2
aggccggcaccttttcctttacccacaacttactttatgaatacgactgactgttttatcgctttggtacaggctatcagagagatcaaacttttgttcaaaggaatacgaaagatggagttcacactgtacaacggtga

SEQ ID NO: 23 - DNA, 139 bp, Foot-and-mouth disease virus SAT 3
aggccggcaccttttccttttatccaacacattttatgaagacaactgactgttttgacgttttgctcgagatctttcacaggttccgacacacgttcaagacagacaggaagatggaattcacactctacaacggtga

SEQ ID NO: 24 - DNA, 139 bp, Foot-and-mouth disease virus SAT 1
aggccggcaccttttcctatttaaaccttgattttatgaagacaactgactgtttcaacgttttgctcgagatccttcacaggttcagacacacattcaagataaatagagagatggaattcacactctacaacggaga

SEQ ID NO: 25 - DNA, 150 bp, Artificial Sequence
  Consensus Sequence
  misc_feature (46)..(46): n is inosine
  misc_feature (68)..(68): n is inosine
gaccggaggccggcrcctttcccttaattacactggacccatgaanacractgactgttttatcgctntggtacacgctatcagagagatcagagcatttttcctaccacgagccacargraaratggaattcacactgcacaacggtga

SEQ ID NO: 26 - DNA, 25 bp, Artificial Sequence
  Synthetic Oligomer
gaccggaggccggcrcctttccctt

SEQ ID NO: 27 - DNA, 33 bp, Artificial Sequence
  Synthetic Oligomer
  misc_feature (9)..(9): n is inosine
  misc_feature (31)..(31): n is inosine
cccatgaanacractgactgttttatcgctntg

SEQ ID NO: 28 - DNA, 20 bp, Artificial Sequence
  Synthetic Oligomer
rgraaratggaattcacact

SEQ ID NO: 29 - DNA, 25 bp, Artificial Sequence
  Synthetic Oligomer
  misc_feature (11)..(11): n is inosine
aagggaaaggngccggcctccggtc

SEQ ID NO: 30 - DNA, 20 bp, Artificial Sequence
  Synthetic Oligomer
  misc_feature (9)..(9): n is inosine
  misc_feature (12)..(12): n is inosine
cagtcagtngtnttcatggg

SEQ ID NO: 31 - DNA, 20 bp, Artificial Sequence
  Synthetic Oligomer
  misc_feature (3)..(3): n is inosine
canagcgataaaacagtcag

SEQ ID NO: 32 - DNA, 20 bp, Artificial Sequence
  Synthetic Oligomer
  misc_feature (15)..(15): n is inosine
  misc_feature (18)..(18): n is inosine
  misc_feature (20)..(20): n is inosine
agtgtgaattccatnttncn

SEQ ID NO: 33 - protein, 13 aa, Artificial Sequence
  Synthetic arginine-rich peptide
  MOD_RES (12)..(12): Xaa is 6-aminohexanoic acid
  MOD_RES (13)..(13): Xaa is beta-alanine
RRRRRRRRRFFXX

SEQ ID NO: 34 - protein, 14 aa, Artificial Sequence
  Synthetic arginine-rich peptide
  MOD_RES (2)..(13): Xaa is 6-aminohexanoic acid
  MOD_RES (14)..(14): Xaa is beta-alanine
RXRRXRRXRRXRXX

SEQ ID NO: 35 - protein, 17 aa, Artificial Sequence
  Synthetic arginine-rich peptide
  MOD_RES (1)..(16): Xaa is 6-aminohexanoic acid
  MOD_RES (17)..(17): Xaa is beta-alanine
RXRXRXRXRXRXRXRXX